SEQUENCE LISTING (199 sequences)

SEQ ID NO: 1 - protein, 113 aa, Artificial Sequence
  xiH
QVQLQQPGAELVRPGASVKLSCKASGFSFTTYWITWVKQRPGQGLEWIGMIHPSDSETRLSQTFKDKATLTVDKSSNTAYMQFSSPTSEDSAVYYCVRDFDVWGAGTTVTVSS

SEQ ID NO: 2 - protein, 113 aa, Artificial Sequence
  zuH3
QVQLVQSGAEVKKPGASVKLSCKASGFSFTTYWITWVRQAPGQGLEWIGMIHPSDSETRLSQTFKDKATLTADKSTNTAYMELSSLRSEDTAVYYCARDFDVWGQGTLVTVSS

SEQ ID NO: 3 - protein, 113 aa, Artificial Sequence
  zuH5
QVQLVQSGAEVKKPGASVKLSCKASGFSFTTYWITWVRQAPGQGLEWIGMIHPSDSETRLSQTFKDKATLTADESTNTAYMELSSLRSEDTAVYYCARDFDVWGQGTLVTVSS

SEQ ID NO: 4 - protein, 113 aa, Artificial Sequence
  zuH3a
QVQLVQSGAEVKKPGASVKLSCKASGFSFTTYWITWVRQAPGQGLEWIGMIHPSDAETRLSQTFKDKATLTADKSTNTAYMELSSLRSEDTAVYYCARDFDVWGQGTLVTVSS

SEQ ID NO: 5 - protein, 113 aa, Artificial Sequence
  zuH3b
QVQLVQSGAEVKKPGASVKVSCKASGFSFTTYWITWVRQAPGQGLEWIGMIHPSDAETRLSQTFKDKATLTADKSTNTAYMELSSLRSEDTAVYYCARDFDVWGQGTLVTVSS

SEQ ID NO: 6 - protein, 113 aa, Artificial Sequence
  zuH3c
QVQLVQSGAEVKKPGASVKVSCKASGFSFTTYWITWVRQAPGQGLEWIGMIHPSDAETRLSQTFKDKATLTADKSTSTAYMELSSLRSEDTAVYYCARDFDVWGQGTLVTVSS

SEQ ID NO: 7 - protein, 113 aa, Artificial Sequence
  zuH3d
QVQLVQSGAEVKKPGASVKVSCKASGFSFTTYWITWVRQAPGQGLEWIGMIHPSDAETRLSQTFKDKATLTADKSTNTAYMELSSLRSEDTAVYYCARDFIVWGQGTLVTVSS

SEQ ID NO: 8 - protein, 113 aa, Artificial Sequence
  zuH3e
QVQLVQSGAEVKKPGASVKVSCKASGFHFTTYWITWVRQAPGQGLEWIGMIHPSDAETRLSQTFKDKATLTADKSTNTAYMELSSLRSEDTAVYYCARDFDVWGQGTLVTVSS

SEQ ID NO: 9 - protein, 113 aa, Artificial Sequence
  zuH3f
QVQLVQSGAEVKKPGASVKVSCKASGFSFRTYWITWVRQAPGQGLEWIGMIHPSDAETRLSQTFKDKATLTADKSTNTAYMELSSLRSEDTAVYYCARDFDVWGQGTLVTVSS

SEQ ID NO: 10 - protein, 112 aa, Artificial Sequence
  xiL
DVLMTQTPLSLPVSLGDQASISCRSSQNIVHNNGNTYLGWYLQKPGQSPKLLIFKVSNRFSGVPDRFSGSGSGTDFTLKISRVEAEDLGVYYCFQGSHIPRTFGGGTRLEIK

SEQ ID NO: 11 - protein, 112 aa, Artificial Sequence
  zuL2
DIQMTQSPSSLSASVGDRVTITCRSSQNIVHNNGNTYLGWYQQKPGKAPKLLIFKVSNRFSGVPSRFSGSGSGTDFTLTISSLQPEDFATYYCFQGSHIPRTFGQGTKVEIK

SEQ ID NO: 12 - protein, 112 aa, Artificial Sequence
  zuL3
DVVMTQTPLSLPVTLGQPASISCRSSQNIVHNNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 13 - protein, 112 aa, Artificial Sequence
  zuL4
DVVMTQTPLSLPVTPGEPASISCRSSQNIVHNNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 14 - protein, 112 aa, Artificial Sequence
  zuL5
DVVMTQSPLSLPVTLGQPASISCRSSQNIVHNNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 15 - protein, 112 aa, Artificial Sequence
  zuL5a
DVVMTQSPLSLPVTLGQPASISCRSSQNIVHQNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 16 - protein, 112 aa, Artificial Sequence
  zuL5b
DVVMTQSPLSLPVTLGQPASISCRSSQNIVHNQGNTYLGWYLQKPGQSPQLLIFKVSNRFSGVPDRFSGSGSGTDFTLKISRVEAEDVGVYYCFQGSHIPRTFGQGTRLEIK

SEQ ID NO: 17 - protein, 112 aa, Artificial Sequence
  zuL5c
DVVMTQSPLSLPVTLGQPASISCRSSQNIVHNNGQTYLGWYLQKPGQSPQLLIFKVSNRFSGVPDRFSGSGSGTDFTLKISRVEAEDVGVYYCFQGSHIPRTFGQGTRLEIK

SEQ ID NO: 18 - protein, 112 aa, Artificial Sequence
  zuL5d
DVVMTQSPLSLPVTLGQPASISCRSSQNIVHNNGNTYLGWYLQKPGQSPQLLIFRVSNRFSGVPDRFSGSGSGTDFTLKISRVEAEDVGVYYCFQGSHIPRTFGQGTRLEIK

SEQ ID NO: 19 - protein, 112 aa, Artificial Sequence
  zuL5e
DVVMTQSPLSLPVTLGQPASISCRSSQNIVHQNAQTYLGWYLQKPGQSPQLLIFKVSNRFSGVPDRFSGSGSGTDFTLKISRVEAEDVGVYYCFQGSHIPRTFGQGTRLEIK

SEQ ID NO: 20 - protein, 112 aa, Artificial Sequence
  zuL5f
DVVMTQSPLSLPVTLGQPASISCRSSQNIVHQNAQTYLGWYLQKPGQSPQLLIFRVSNRFSGVPDRFSGSGSGTDFTLKISRVEAEDVGVYYCFQGSHIPRTFGQGTRLEIK

SEQ ID NO: 21 - protein, 112 aa, Artificial Sequence
  zuL5g
DVVMTQSPLSLPVTLGQPASISCRSSQNIVHQNGQTYLGWYLQKPGQSPQLLIFRVSNRFSGVPDRFSGSGSGTDFTLKISRVEAEDVGVYYCFQGSHIPRTFGQGTRLEIK

SEQ ID NO: 22 - protein, 112 aa, Artificial Sequence
  zuL5h
DVVMTQSPLSLPVTLGQPASISCRSSQNIVHQHGQTYLGWYLQKPGQSPQLLIFRVSNRFSGVPDRFSGSGSGTDFTLKISRVEAEDVGVYYCFQGSHIPRTFGQGTRLEIK

SEQ ID NO: 23 - protein, 112 aa, Artificial Sequence
  zuL5i
DVVMTQSPLSLPVTLGQPASISCRSSQNIVHQTGQTYLGWYLQKPGQSPQLLIFRVSNRFSGVPDRFSGSGSGTDFTLKISRVEAEDVGVYYCFQGSHIPRTFGQGTRLEIK

SEQ ID NO: 24 - protein, 112 aa, Artificial Sequence
  zuL5j
DVVMTQSPLSLPVTLGQPASISCRSSQNIVHQNRQTYLGWYLQKPGQSPQLLIFRVSNRFSGVPDRFSGSGSGTDFTLKISRVEAEDVGVYYCFQGSHIPRTFGQGTRLEIK

SEQ ID NO: 25 - protein, 112 aa, Artificial Sequence
  zuL5k
DVVMTQSPLSLPVTLGQPASISCRSSQNIVHQNAGTYLGWYLQKPGQSPQLLIFRVSNRFSGVPDRFSGSGSGTDFTLKISRVEAEDVGVYYCFQGSHIPRTFGQGTRLEIK

SEQ ID NO: 26 - protein, 111 aa, Artificial Sequence
  zuL5L
DVVMTQSPLSLPVTLGQPASISCRSSQNIVHQNQTYLGWYLQKPGQSPQLLIFRVSNRFSGVPDRFSGSGSGTDFTLKISRVEAEDVGVYYCFQGSHIPRTFGQGTRLEIK

SEQ ID NO: 27 - protein, 111 aa, Artificial Sequence
  zuL5m
DVVMTQSPLSLPVTLGQPASISCRSSQNIVHQGQTYLGWYLQKPGQSPQLLIFRVSNRFSGVPDRFSGSGSGTDFTLKISRVEAEDVGVYYCFQGSHIPRTFGQGTRLEIK

SEQ ID NO: 28 - protein, 111 aa, Artificial Sequence
  zuL5n
DVVMTQSPLSLPVTLGQPASISCRSSQNIVHQQGTYLGWYLQKPGQSPQLLIFRVSNRFSGVPDRFSGSGSGTDFTLKISRVEAEDVGVYYCFQGSHIPRTFGQGTRLEIK

SEQ ID NO: 29 - protein, 112 aa, Artificial Sequence
  zuL5o
DVVMTQSPLSLPVTLGQPASISCRSSQNIVHQNGQTYLGWYLQKPGQSPQLLIFRVSNRFSGVPDRFSGSGSGTDFTLKISRVEAEDVGVYYCFQGSHNPRTFGQGTRLEIK

SEQ ID NO: 30 - protein, 112 aa, Artificial Sequence
  zuL5p
DVVMTQSPLSLPVTLGQPASISCRSSQNIVHQNGQTYLGWYLQKPGQSPQLLIFRVSNRFSGVPDRFSGSGSGTDFTLKISRVEAEDVGVYYCFQGSHHPRTFGQGTRLEIK

SEQ ID NO: 31 - protein, 112 aa, Artificial Sequence
  zuL5q
DVVMTQSPLSLPVTLGQPASISCRSSQNIVHQNGQTYLGWYLQKPGQSPQLLIFRVSNRFLGVPDRFSGSGSGTDFTLKISRVEAEDVGVYYCFQGSHIPRTFGQGTRLEIK

SEQ ID NO: 32 - protein, 25 aa, Artificial Sequence
  H-FR1
  MISC_FEATURE (5)..(5): Xaa = Q or V
  MISC_FEATURE (7)..(7): Xaa = S or P
  MISC_FEATURE (11)..(11): Xaa = V or L
  MISC_FEATURE (12)..(12): Xaa = V or K
  MISC_FEATURE (13)..(13): Xaa = R or K
  MISC_FEATURE (20)..(20): Xaa = L or V
QVQLXQXGAEXXXPGASVKXSCKAS

SEQ ID NO: 33 - protein, 25 aa, Artificial Sequence
  H-FR1-2
  MISC_FEATURE (20)..(20): X = V or L
QVQLVQSGAEVKKPGASVKXSCKAS

SEQ ID NO: 34 - protein, 25 aa, Artificial Sequence
  H-FR1-3
QVQLQQPGAELVRPGASVKLSCKAS

SEQ ID NO: 35 - protein, 25 aa, Artificial Sequence
  H-FR1-4
QVQLVQSGAEVKKPGASVKLSCKAS

SEQ ID NO: 36 - protein, 25 aa, Artificial Sequence [sequence omitted]

SEQ ID NO: 37 - protein, 10 aa, Artificial Sequence
  H-CDR1-1
  MISC_FEATURE (5)..(5): Xaa = T or R
GFSFXTYWIT

SEQ ID NO: 38 - protein, 10 aa, Artificial Sequence
  H-CDR1-2
GFSFTTYWIT

SEQ ID NO: 39 - protein, 10 aa, Artificial Sequence
  H-CDR1-3
GFSFRTYWIT

SEQ ID NO: 40 - protein, 14 aa, Artificial Sequence
  H-FR2-1
  MISC_FEATURE (3)..(3): Xaa = K or R
  MISC_FEATURE (5)..(5): Xaa = R or A
WVXQXPGQGLEWIG

SEQ ID NO: 41 - protein, 14 aa, Artificial Sequence
  H-FR2-2
WVKQRPGQGLEWIG

SEQ ID NO: 42 - protein, 14 aa, Artificial Sequence
  H-FR2-3
WVRQAPGQGLEWIG

SEQ ID NO: 43 - protein, 17 aa, Artificial Sequence
  H-CDR2-1
  MISC_FEATURE (7)..(7): X = A or S
MIHPSDXETRLSQTFKD

SEQ ID NO: 44 - protein, 17 aa, Artificial Sequence
  H-CDR2-2
MIHPSDSETRLSQTFKD

SEQ ID NO: 45 - protein, 17 aa, Artificial Sequence
  H-CDR2-3
MIHPSDAETRLSQTFKD

SEQ ID NO: 46 - protein, 32 aa, Artificial Sequence
  H-FR3-1
  MISC_FEATURE (6)..(6): Xaa = V or A
  MISC_FEATURE (8)..(8): Xaa = K or E
  MISC_FEATURE (10)..(10): Xaa = S or T
  MISC_FEATURE (11)..(11): Xaa = N or S
  MISC_FEATURE (16)..(16): Xaa = E or Q
  MISC_FEATURE (20)..(20): Xaa = P or L
  MISC_FEATURE (21)..(21): Xaa = T or R
  MISC_FEATURE (25)..(25): Xaa = S or T
  MISC_FEATURE (31)..(31): Xaa = V or A
KATLTXDXSXXTAYMXFSSXXSEDXAVYYCXR

SEQ ID NO: 47 - protein, 32 aa, Artificial Sequence
  H-FR3-2
KATLTVDKSSNTAYMQFSSPTSEDSAVYYCVR

SEQ ID NO: 48 - protein, 32 aa, Artificial Sequence
  H-FR3-3
KATLTADKSTNTAYMELSSLRSEDTAVYYCAR

SEQ ID NO: 49 - protein, 32 aa, Artificial Sequence
  H-FR3-4
KATLTADESTNTAYMELSSLRSEDTAVYYCAR

SEQ ID NO: 50 - protein, 32 aa, Artificial Sequence
  H-FR3-5
KATLTADKSTSTAYMELSSLRSEDTAVYYCAR

SEQ ID NO: 51 - protein, 4 aa, Artificial Sequence
  H-CDR3-1
  MISC_FEATURE (1)..(1): Xaa = A or V
DFDV

SEQ ID NO: 52 - protein, 4 aa, Artificial Sequence
  H-CDR3-2
DFDV

SEQ ID NO: 53 - protein, 4 aa, Artificial Sequence
  H-CDR3-3
DFDV

SEQ ID NO: 54 - protein, 11 aa, Artificial Sequence
  H-FR4-1
  MISC_FEATURE (3)..(3): Xaa = A or G
  MISC_FEATURE (6)..(6): Xaa = T or L
WGXGTXVTVSS

SEQ ID NO: 55 - protein, 11 aa, Artificial Sequence
  H-FR4-2
WGAGTTVTVSS

SEQ ID NO: 56 - protein, 11 aa, Artificial Sequence
  H-FR4-3
WGQGTLVTVSS

SEQ ID NO: 57 - protein, 23 aa, Artificial Sequence
  L-FR1-1
  MISC_FEATURE (3)..(3): Xaa = L or V
  MISC_FEATURE (7)..(7): Xaa = T or S
  MISC_FEATURE (14)..(14): Xaa = S or T
  MISC_FEATURE (15)..(15): Xaa = L or P
  MISC_FEATURE (17)..(17): Xaa = D, Q, or E
  MISC_FEATURE (18)..(18): Xaa = Q or P
DVXMTQXPLSLPVXXGXXASISC

SEQ ID NO: 58 - protein, 23 aa, Artificial Sequence
  L-FR1-2
DVLMTQTPLSLPVSLGDQASISC

SEQ ID NO: 59 - protein, 23 aa, Artificial Sequence
  L-FR1-3
DVVMTQTPLSLPVTLGQPASISC

SEQ ID NO: 60 - protein, 23 aa, Artificial Sequence
  L-FR1-4
DVVMTQTPLSLPVTPGEPASISC

SEQ ID NO: 61 - protein, 23 aa, Artificial Sequence
  L-FR1-5
DVVMTQSPLSLPVTLGQPASISC

SEQ ID NO: 62 - protein, 16 aa, Artificial Sequence
  L-CDR1-1
  MISC_FEATURE (9)..(9): Xaa = N or Q
  MISC_FEATURE (10)..(10): Xaa = N or T
  MISC_FEATURE (11)..(11): Xaa = A, R, or G
  MISC_FEATURE (12)..(12): Xaa = N, Q, or G
RSSQNIVHXXXXTYLG

SEQ ID NO: 63 - protein, 16 aa, Artificial Sequence
  L-CDR1-2
  MISC_FEATURE (10)..(10): Xaa = N or T
  MISC_FEATURE (11)..(11): Xaa = A, R, or G
RSSQNIVHQXXQTYLG

SEQ ID NO: 64 - protein, 16 aa, Artificial Sequence
  L-CDR1-3
RSSQNIVHNNGNTYLG

SEQ ID NO: 65 - protein, 16 aa, Artificial Sequence
  L-CDR1-4
RSSQNIVHQNGNTYLG

SEQ ID NO: 66 - protein, 16 aa, Artificial Sequence
  L-CDR1-5
RSSQNIVHNNGQTYLG

SEQ ID NO: 67 - protein, 20 aa, Artificial Sequence
  L-CDR1-6
RSSQNIVHQNAQTYLGWYLQ

SEQ ID NO: 68 - protein, 16 aa, Artificial Sequence
  L-CDR1-7
RSSQNIVHQNGQTYLG

SEQ ID NO: 69 - protein, 16 aa, Artificial Sequence
  L-CDR1-8
RSSQNIVHQTGQTYLG

SEQ ID NO: 70 - protein, 16 aa, Artificial Sequence
  L-CDR1-9
RSSQNIVHQNRQTYLG

SEQ ID NO: 71 - protein, 16 aa, Artificial Sequence
  L-CDR1-10
RSSQNIVHQNAGTYLG

SEQ ID NO: 72 - protein, 14 aa, Artificial Sequence
  L-FR2-1
  MISC_FEATURE (11)..(11): X = K or Q
WYLQKPGQSPXLLI

SEQ ID NO: 73 - protein, 14 aa, Artificial Sequence
  L-FR2-2
WYLQKPGQSPKLLI

SEQ ID NO: 74 - protein, 14 aa, Artificial Sequence
  L-FR2-3
WYLQKPGQSPQLLI

SEQ ID NO: 75 - protein, 8 aa, Artificial Sequence
  L-CDR2-1
  MISC_FEATURE (2)..(2): Xaa = K or R
  MISC_FEATURE (8)..(8): Xaa = S or L
FXVSNRFX

SEQ ID NO: 76 - protein, 8 aa, Artificial Sequence
  L-CDR2-2
FKVSNRFS

SEQ ID NO: 77 - protein, 8 aa, Artificial Sequence
  L-CDR2-3
FRVSNRFS

SEQ ID NO: 78 - protein, 8 aa, Artificial Sequence
  L-CDR2-4
FRVSNRFL

SEQ ID NO: 79 - protein, 32 aa, Artificial Sequence
  L-FR3-1
  MISC_FEATURE (27)..(27): Xaa = L or V
GVPDRFSGSGSGTDFTLKISRVEAEDXGVYYC

SEQ ID NO: 80 - protein, 32 aa, Artificial Sequence
  L-FR3-2
GVPDRFSGSGSGTDFTLKISRVEAEDLGVYYC

SEQ ID NO: 81 - protein, 32 aa, Artificial Sequence
  L-FR3-3
GVPDRFSGSGSGTDFTLKISRVEAEDVGVYYC

SEQ ID NO: 82 - protein, 9 aa, Artificial Sequence
  L-CDR3-1
  MISC_FEATURE (6)..(6): Xaa = I, N, or H
FQGSHXPRT

SEQ ID NO: 83 - protein, 9 aa, Artificial Sequence
  L-CDR3-2
FQGSHIPRT

SEQ ID NO: 84 - protein, 9 aa, Artificial Sequence
  L-CDR3-3
FQGSHNPRT

SEQ ID NO: 85 - protein, 9 aa, Artificial Sequence
  L-CDR3-4
FQGSHHPRT

SEQ ID NO: 86 - protein, 10 aa, Artificial Sequence
  L-FR4-1
  MISC_FEATURE (3)..(3): Xaa = G or Q
FGXGTRLEIK

SEQ ID NO: 87 - protein, 10 aa, Artificial Sequence
  L-FR4-2
FGGGTRLEIK

SEQ ID NO: 88 - protein, 10 aa, Artificial Sequence
  L-FR4-3
FGQGTRLEIK

SEQ ID NO: 89 - protein, 10 aa, Artificial Sequence
  xiH-CDR1
GFSFTTYWIT

SEQ ID NO: 90 - protein, 17 aa, Artificial Sequence
  xiH-CDR2
MIHPSDSETRLSQTFKD

SEQ ID NO: 91 - protein, 4 aa, Artificial Sequence
  xiH-CDR3
DFDV

SEQ ID NO: 92 - protein, 10 aa, Artificial Sequence
  zuH3-CDR1
GFSFTTYWIT

SEQ ID NO: 93 - protein, 17 aa, Artificial Sequence
  zuH3-CDR2
MIHPSDSETRLSQTFKD

SEQ ID NO: 94 - protein, 4 aa, Artificial Sequence
  zuH3-CDR3
DFDV

SEQ ID NO: 95 - protein, 10 aa, Artificial Sequence
  zuH5-CDR1
GFSFTTYWIT

SEQ ID NO: 96 - protein, 17 aa, Artificial Sequence
  zuH5-CDR2
MIHPSDSETRLSQTFKD

SEQ ID NO: 97 - protein, 4 aa, Artificial Sequence
  zuH5-CDR3
DFDV

SEQ ID NO: 98 - protein, 10 aa, Artificial Sequence
  zuH3a-CDR1
GFSFTTYWIT

SEQ ID NO: 99 - protein, 17 aa, Artificial Sequence
  zuH3a-CDR2
MIHPSDAETRLSQTFKD

SEQ ID NO: 100 - protein, 4 aa, Artificial Sequence
  zuH3a-CDR3
DFDV

SEQ ID NO: 101 - protein, 10 aa, Artificial Sequence
  zuH3b-CDR1
GFSFTTYWIT

SEQ ID NO: 102 - protein, 17 aa, Artificial Sequence
  zuH3b-CDR2
MIHPSDAETRLSQTFKD

SEQ ID NO: 103 - protein, 4 aa, Artificial Sequence
  zuH3b-CDR3
DFDV

SEQ ID NO: 104 - protein, 10 aa, Artificial Sequence
  zuH3c-CDR1
GFSFTTYWIT

SEQ ID NO: 105 - protein, 17 aa, Artificial Sequence
  zuH3c-CDR2
MIHPSDAETRLSQTFKD

SEQ ID NO: 106 - protein, 4 aa, Artificial Sequence
  zuH3c-CDR3
DFDV

SEQ ID NO: 107 - protein, 10 aa, Artificial Sequence
  zuH3d-CDR1
GFSFTTYWIT

SEQ ID NO: 108 - protein, 17 aa, Artificial Sequence
  zuH3d-CDR2
MIHPSDAETRLSQTFKD

SEQ ID NO: 109 - protein, 4 aa, Artificial Sequence
  zuH3d-CDR3
DFIV

SEQ ID NO: 110 - protein, 10 aa, Artificial Sequence
  zuH3e-CDR1
GFHFTTYWIT

SEQ ID NO: 111 - protein, 17 aa, Artificial Sequence
  zuH3e-CDR2
MIHPSDAETRLSQTFKD

SEQ ID NO: 112 - protein, 4 aa, Artificial Sequence
  zuH3e-CDR3
DFDV

SEQ ID NO: 113 - protein, 10 aa, Artificial Sequence
  zuH3f-CDR1
GFSFRTYWIT

SEQ ID NO: 114 - protein, 17 aa, Artificial Sequence
  zuH3f-CDR2
MIHPSDAETRLSQTFKD

SEQ ID NO: 115 - protein, 4 aa, Artificial Sequence
  zuH3f-CDR3
DFDV

SEQ ID NO: 116 - protein, 16 aa, Artificial Sequence
  xiL-CDR1
RSSQNIVHNNGNTYLG

SEQ ID NO: 117 - protein, 8 aa, Artificial Sequence
  xiL-CDR2
FKVSNRFS

SEQ ID NO: 118 - protein, 9 aa, Artificial Sequence
  xiL-CDR3
FQGSHIPRT

SEQ ID NO: 119 - protein, 16 aa, Artificial Sequence
  zuL2-CDR1
RSSQNIVHNNGNTYLG

SEQ ID NO: 120 - protein, 8 aa, Artificial Sequence
  zuL2-CDR2
FKVSNRFS

SEQ ID NO: 121 - protein, 9 aa, Artificial Sequence
  zuL2-CDR3
FQGSHIPRT

SEQ ID NO: 122 - protein, 16 aa, Artificial Sequence
  zuL3-CDR1
RSSQNIVHNNGNTYLG

SEQ ID NO: 123 - protein, 8 aa, Artificial Sequence
  zuL3-CDR2
FKVSNRFS

SEQ ID NO: 124 - protein, 9 aa, Artificial Sequence
  zuL3-CDR3
FQGSHIPRT

SEQ ID NO: 125 - protein, 16 aa, Artificial Sequence
  zuL4-CDR1
RSSQNIVHNNGNTYLG

SEQ ID NO: 126 - protein, 8 aa, Artificial Sequence
  zuL4-CDR2
FKVSNRFS

SEQ ID NO: 127 - protein, 9 aa, Artificial Sequence
  zuL4-CDR3
FQGSHIPRT

SEQ ID NO: 128 - protein, 16 aa, Artificial Sequence
  zuL5-CDR1
RSSQNIVHNNGNTYLG

SEQ ID NO: 129 - protein, 8 aa, Artificial Sequence
  zuL5-CDR2
FKVSNRFS

SEQ ID NO: 130 - protein, 9 aa, Artificial Sequence
  zuL5-CDR3
FQGSHIPRT

SEQ ID NO: 131 - protein, 16 aa, Artificial Sequence
  zuL5a-CDR1
RSSQNIVHQNGNTYLG

SEQ ID NO: 132 - protein, 8 aa, Artificial Sequence
  zuL5a-CDR2
FKVSNRFS

SEQ ID NO: 133 - protein, 9 aa, Artificial Sequence
  zuL5a-CDR3
FQGSHIPRT

SEQ ID NO: 134 - protein, 16 aa, Artificial Sequence
  zuL5b-CDR1
RSSQNIVHNQGNTYLG

SEQ ID NO: 135 - protein, 8 aa, Artificial Sequence
  zuL5b-CDR2
FKVSNRFS

SEQ ID NO: 136 - protein, 9 aa, Artificial Sequence
  zuL5b-CDR3
FQGSHIPRT

SEQ ID NO: 137 - protein, 16 aa, Artificial Sequence
  zuL5c-CDR1
RSSQNIVHNNGQTYLG

SEQ ID NO: 138 - protein, 8 aa, Artificial Sequence
  zuL5c-CDR2
FKVSNRFS

SEQ ID NO: 139 - protein, 9 aa, Artificial Sequence
  zuL5c-CDR3
FQGSHIPRT

SEQ ID NO: 140 - protein, 16 aa, Artificial Sequence
  zuL5d-CDR1
RSSQNIVHNNGNTYLG

SEQ ID NO: 141 - protein, 8 aa, Artificial Sequence
  zuL5d-CDR2
FRVSNRFS

SEQ ID NO: 142 - protein, 9 aa, Artificial Sequence
  zuL5d-CDR3
FQGSHIPRT

SEQ ID NO: 143 - protein, 16 aa, Artificial Sequence
  zuL5e-CDR1
RSSQNIVHQNAQTYLG

SEQ ID NO: 144 - protein, 8 aa, Artificial Sequence
  zuL5e-CDR2
FKVSNRFS

SEQ ID NO: 145 - protein, 9 aa, Artificial Sequence
  zuL5e-CDR3
FQGSHIPRT

SEQ ID NO: 146 - protein, 16 aa, Artificial Sequence
  zuL5f-CDR1
RSSQNIVHQNAQTYLG

SEQ ID NO: 147 - protein, 8 aa, Artificial Sequence
  zuL5f-CDR2
FRVSNRFS

SEQ ID NO: 148 - protein, 9 aa, Artificial Sequence
  zuL5f-CDR3
FQGSHIPRT

SEQ ID NO: 149 - protein, 16 aa, Artificial Sequence
  zuL5g-CDR1
RSSQNIVHQNGQTYLG

SEQ ID NO: 150 - protein, 8 aa, Artificial Sequence
  zuL5g-CDR2
FRVSNRFS

SEQ ID NO: 151 - protein, 9 aa, Artificial Sequence
  zuL5g-CDR3
FQGSHIPRT

SEQ ID NO: 152 - protein, 16 aa, Artificial Sequence
  zuL5h-CDR1
RSSQNIVHQHGQTYLG

SEQ ID NO: 153 - protein, 8 aa, Artificial Sequence
  zuL5h-CDR2
FRVSNRFS

SEQ ID NO: 154 - protein, 9 aa, Artificial Sequence
  zuL5h-CDR3
FQGSHIPRT

SEQ ID NO: 155 - protein, 16 aa, Artificial Sequence
  zuL5i-CDR1
RSSQNIVHQTGQTYLG

SEQ ID NO: 156 - protein, 8 aa, Artificial Sequence
  zuL5i-CDR2
FRVSNRFS

SEQ ID NO: 157 - protein, 9 aa, Artificial Sequence
  zuL5i-CDR3
FQGSHIPRT

SEQ ID NO: 158 - protein, 16 aa, Artificial Sequence
  zuL5j-CDR1
RSSQNIVHQNRQTYLG

SEQ ID NO: 159 - protein, 8 aa, Artificial Sequence
  zuL5j-CDR2
FRVSNRFS

SEQ ID NO: 160 - protein, 9 aa, Artificial Sequence
  zuL5j-CDR3
FQGSHIPRT

SEQ ID NO: 161 - protein, 16 aa, Artificial Sequence
  zuL5k-CDR1
RSSQNIVHQNAGTYLG

SEQ ID NO: 162 - protein, 8 aa, Artificial Sequence
  zuL5k-CDR2
FRVSNRFS

SEQ ID NO: 163 - protein, 9 aa, Artificial Sequence
  zuL5k-CDR3
FQGSHIPRT

SEQ ID NO: 164 - protein, 15 aa, Artificial Sequence
  zuL5L-CDR1
RSSQNIVHQNQTYLG

SEQ ID NO: 165 - protein, 8 aa, Artificial Sequence
  zuL5L-CDR2
FRVSNRFS

SEQ ID NO: 166 - protein, 9 aa, Artificial Sequence
  zuL5L-CDR3
FQGSHIPRT

SEQ ID NO: 167 - protein, 15 aa, Artificial Sequence
  zuL5m-CDR1
RSSQNIVHQGQTYLG

SEQ ID NO: 168 - protein, 8 aa, Artificial Sequence
  zuL5m-CDR2
FRVSNRFS

SEQ ID NO: 169 - protein, 9 aa, Artificial Sequence
  zuL5m-CDR3
FQGSHIPRT

SEQ ID NO: 170 - protein, 15 aa, Artificial Sequence
  zuL5n-CDR1
RSSQNIVHQQGTYLG

SEQ ID NO: 171 - protein, 8 aa, Artificial Sequence
  zuL5n-CDR2
FRVSNRFS

SEQ ID NO: 172 - protein, 9 aa, Artificial Sequence
  zuL5n-CDR3
FQGSHIPRT

SEQ ID NO: 173 - protein, 16 aa, Artificial Sequence
  zuL5o-CDR1
RSSQNIVHQNGQTYLG

SEQ ID NO: 174 - protein, 8 aa, Artificial Sequence
  zuL5o-CDR2
FRVSNRFS

SEQ ID NO: 175 - protein, 9 aa, Artificial Sequence
  zuL5o-CDR3
FQGSHNPRT

SEQ ID NO: 176 - protein, 16 aa, Artificial Sequence
  zuL5p-CDR1
RSSQNIVHQNGQTYLG

SEQ ID NO: 177 - protein, 8 aa, Artificial Sequence
  zuL5p-CDR2
FRVSNRFS

SEQ ID NO: 178 - protein, 9 aa, Artificial Sequence
  zuL5p-CDR3
FQGSHHPRT

SEQ ID NO: 179 - protein, 16 aa, Artificial Sequence
  zuL5q-CDR1
RSSQNIVHQNGQTYLG

SEQ ID NO: 180 - protein, 8 aa, Artificial Sequence
  zuL5q-CDR2
FRVSNRFL

SEQ ID NO: 181 - protein, 9 aa, Artificial Sequence
  zuL5q-CDR3
FQGSHIPRT

SEQ ID NO: 182 - protein, 11 aa, Artificial Sequence
  HEG1 fragment
SKSPSLVSLPT

SEQ ID NO: 183 - protein, 111 aa, Artificial Sequence
  SLURPgpi
GSLKCYTCKEPMTSASCRTITRCKPEDTACMTTLVTVEAEYPFNQSPVVTRSCSSSCVATDPDSIGAAHLIFCCFRDLCNSELSRDGAPSLGSPGGLLLALALFLLLGVLL

SEQ ID NO: 184 - protein, 110 aa, Artificial Sequence
  IgG1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 185 - protein, 11 aa, Artificial Sequence
  IgG1 hinge region
CPPCPAPELLG

SEQ ID NO: 186 - protein, 11 aa, Artificial Sequence
  IgG4 hinge region
CPSCPAPEFLG

SEQ ID NO: 187 - protein, 11 aa, Artificial Sequence
  IgG4PE hinge region
CPPCPAPEFEG

SEQ ID NO: 188 - protein, 104 aa, Artificial Sequence
  IgG1 CH2 region
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 189 - protein, 104 aa, Artificial Sequence
  IgG4 or IgG4PE CH2 region
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 190 - protein, 19 aa, Artificial Sequence
  Heavy chain signal sequence VH1
MDWTWRILFLVAAATGAHS

SEQ ID NO: 191 - protein, 19 aa, Artificial Sequence
  Heavy chain signal sequence VH2
MDILCSTLLLLTVPSWVLS

SEQ ID NO: 192 - protein, 19 aa, Artificial Sequence
  Heavy chain signal sequence VH3
MEFGLSWVFLVALLRGVQC

SEQ ID NO: 193 - protein, 19 aa, Artificial Sequence
  Heavy chain signal sequence VH4
MKHLWFFLLLVAAPRWVLS

SEQ ID NO: 194 - protein, 19 aa, Artificial Sequence
  Heavy chain signal sequence VH5
MGSTAILALLLAVLQGVCA

SEQ ID NO: 195 - protein, 20 aa, Artificial Sequence
  Heavy chain signal sequence VH6
MSVSFLIFLPVLGLPWGVLS

SEQ ID NO: 196 - protein, 22 aa, Artificial Sequence
  Light chain signal sequence KL1
MDMRVPAQLLGLLLLWVPGARC

SEQ ID NO: 197 - protein, 20 aa, Artificial Sequence
  Light chain signal sequence KL2
MRLPAQLLGLLMLWVPGSSG

SEQ ID NO: 198 - protein, 19 aa, Artificial Sequence
  Mouse heavy chain signal sequence
MGWSSIILFLVATTTGVHS

SEQ ID NO: 199 - protein, 19 aa, Artificial Sequence
  Mouse light chain signal sequence
MKLPVRLLVLMFWIPASIS

(19) United States
(12) Patent Application Publication
(10) Pub. No.: US 2025/0092152 A1
(43) Pub. Date: Mar. 20, 2025

(54) HUMANIZED ANTIBODY CAPABLE OF BINDING TO HEG1 PROTEIN

(71) Applicants: Kanagawa Prefectural Hospital Organization, Yokohama-shi (JP); The University of Tokyo, Tokyo (JP)

(72) Inventors: Shoutaro TSUJI, Yokohama-shi (JP); Kohzoh IMAI, Tokyo (JP)

(21) Appl. No.: 18/574,969

(22) PCT Filed: Jun. 30, 2022
(86) PCT No.: PCT/JP2022/026140

§ 371 Date: Nov. 14, 2024

(30) Foreign Application Priority Data
Jun. 30, 2021 (JP) 2021-108506

(51) Int. Cl. C07K 16/30

(52) U.S. Cl. CPC C07K 16/30; C07K 2317/24; C07K 2317/565; C07K 2317/567

(57) ABSTRACT

The present invention provides a humanized antibody or an antigen-binding fragment thereof that binds to a HEG1 protein, in which the antibody is capable of binding to a human HEG1 protein expressed in mesothelioma cells.

### TECHNICAL FIELD

The present disclosure provides a humanized antibody that binds to a HEG1 protein.

### BACKGROUND ART

Malignant mesothelioma is a disease that has become a major social problem as a malignant tumor that is primarily caused by exposure to asbestos. Early detection is difficult and it is positioned as one of malignant tumors with poor prognosis. Malignant mesothelioma may be pathologically similar to metastatic adenocarcinoma, sarcoma, and reactive mesothelial cells, which are benign growths, and is often difficult to pathologically differentiate. In addition, it is not rare that various tissue types such as epithelial type and sarcoma type are taken, and diagnosis is difficult.

HEG1 protein has been shown to be effective as a marker for the diagnosis of malignant mesothelioma (Patent Literature 1). Patent Literature 1 discloses that mesothelioma can be detected with high sensitivity and specificity by reacting a mouse antibody that binds to a HEG1 protein with mesothelioma tissue.

### CITATION LIST

### Patent Literature

Patent Literature 1: WO2017/141604

### SUMMARY OF INVENTION

The present disclosure provides a humanized antibody that binds to a HEG1 protein.

The present inventors have developed humanized antibodies that bind to HEG1 proteins. The humanized antibodies are suitable for use in pharmaceutical applications.

According to the present invention, the following invention is provided.

1: An antibody or an antigen-binding fragment thereof, in which
the antibody is a humanized antibody capable of binding to a human HEG1 protein expressed on mesothelioma cells, and
the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 62, a light chain CDR2 having an amino acid sequence set forth in SEQ ID NO: 75, and a light chain CDR3 having an amino acid sequence set forth in SEQ ID NO: 82.

2: The antibody or the antigen-binding fragment thereof according to above 1, in which the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 63, a light chain CDR2 having an amino acid sequence set forth in SEQ ID NO: 75, and a light chain CDR3 having an amino acid sequence set forth in SEQ ID NO: 82.

3: The antibody or the antigen-binding fragment thereof according to above 1 or 2, in which
the heavy chain variable region includes:
(1) a heavy chain CDR1 having an amino acid sequence set forth in SEQ ID NO: 89, a heavy chain CDR2 having an amino acid sequence set forth in SEQ ID NO: 90, and a heavy chain CDR3 having an amino acid sequence set forth in SEQ ID NO: 91,
(2) a heavy chain CDR1 having an amino acid sequence set forth in SEQ ID NO: 92, a heavy chain CDR2 having an amino acid sequence set forth in SEQ ID NO: 93, and a heavy chain CDR3 having an amino acid sequence set forth in SEQ ID NO: 94,
(3) a heavy chain CDR1 having an amino acid sequence set forth in SEQ ID NO: 95, a heavy chain CDR2 having an amino acid sequence set forth in SEQ ID NO: 96, and a heavy chain CDR3 having an amino acid sequence set forth in SEQ ID NO: 97,
(4) a heavy chain CDR1 having an amino acid sequence set forth in SEQ ID NO: 98, a heavy chain CDR2 having an amino acid sequence set forth in SEQ ID NO: 99, and a heavy chain CDR3 having an amino acid sequence set forth in SEQ ID NO: 100,
(5) a heavy chain CDR1 having an amino acid sequence set forth in SEQ ID NO: 101, a heavy chain CDR2 having an amino acid sequence set forth in SEQ ID NO: 102, and a heavy chain CDR3 having an amino acid sequence set forth in SEQ ID NO: 103,
(6) a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106,
(7) a heavy chain CDR1 having an amino acid sequence set forth in SEQ ID NO: 110, a heavy chain CDR2 having an amino acid sequence set forth in SEQ ID NO: 111, and a heavy chain CDR3 having an amino acid sequence set forth in SEQ ID NO: 112, or
(8) a heavy chain CDR1 having an amino acid sequence set forth in SEQ ID NO: 113, a heavy chain CDR2 having an amino acid sequence set forth in SEQ ID NO: 114, and a heavy chain CDR3 having an amino acid sequence set forth in SEQ ID NO: 115.

4: The antibody or the antigen-binding fragment thereof according to any one of above 1 to 3, in which
the light chain variable region includes
(1) a light chain CDR1 having an amino acid sequence set forth in SEQ ID NO: 116, a light chain CDR2 having an amino acid sequence set forth in SEQ ID NO: 117, and a light chain CDR3 having an amino acid sequence set forth in SEQ ID NO: 118,
(2) a light chain CDR1 having an amino acid sequence set forth in SEQ ID NO: 119, a light chain CDR2 having an amino acid sequence set forth in SEQ ID NO: 120, and a light chain CDR3 having an amino acid sequence set forth in SEQ ID NO: 121,
(3) a light chain CDR1 having an amino acid sequence set forth in SEQ ID NO: 122, a light chain CDR2 having an amino acid sequence set forth in SEQ ID NO: 123, and a light chain CDR3 having an amino acid sequence set forth in SEQ ID NO: 124,
(4) a light chain CDR1 having an amino acid sequence set forth in SEQ ID NO: 128, a light chain CDR2 having an amino acid sequence set forth in SEQ ID NO: 129, and a light chain CDR3 having an amino acid sequence set forth in SEQ ID NO: 130,
(5) a light chain CDR1 having an amino acid sequence set forth in SEQ ID NO: 131, a light chain CDR2 having an amino acid sequence set forth in SEQ ID NO: 132, and a light chain CDR3 having an amino acid sequence set forth in SEQ ID NO: 133,
(6) a light chain CDR1 having an amino acid sequence set forth in SEQ ID NO: 134, a light chain CDR2 having an amino acid sequence set forth in SEQ ID NO: 135, and a light chain CDR3 having an amino acid sequence set forth in SEQ ID NO: 136,
(7) a light chain CDR1 having an amino acid sequence set forth in SEQ ID NO: 137, a light chain CDR2 having an amino acid sequence set forth in SEQ ID NO: 138, and a light chain CDR3 having an amino acid sequence set forth in SEQ ID NO: 139,
(8) a light chain CDR1 having an amino acid sequence set forth in SEQ ID NO: 140, a light chain CDR2 having an amino acid sequence set forth in SEQ ID NO: 141, and a light chain CDR3 having an amino acid sequence set forth in SEQ ID NO: 142,
(9) a light chain CDR1 having an amino acid sequence set forth in SEQ ID NO: 143, a light chain CDR2 having an amino acid sequence set forth in SEQ ID NO: 144, and a light chain CDR3 having an amino acid sequence set forth in SEQ ID NO: 145,
(10) a light chain CDR1 having an amino acid sequence set forth in SEQ ID NO: 146, a light chain CDR2 having an amino acid sequence set forth in SEQ ID NO: 147, and a light chain CDR3 having an amino acid sequence set forth in SEQ ID NO: 148,
(11) a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151,
(12) a light chain CDR1 having an amino acid sequence set forth in SEQ ID NO: 152, a light chain CDR2 having an amino acid sequence set forth in SEQ ID NO: 153, and a light chain CDR3 having an amino acid sequence set forth in SEQ ID NO: 154,
(13) a light chain CDR1 having an amino acid sequence set forth in SEQ ID NO: 155, a light chain CDR2 having an amino acid sequence set forth in SEQ ID NO: 156, and a light chain CDR3 having an amino acid sequence set forth in SEQ ID NO: 157,
(14) a light chain CDR1 having an amino acid sequence set forth in SEQ ID NO: 158, a light chain CDR2 having an amino acid sequence set forth in SEQ ID NO: 159, and a light chain CDR3 having an amino acid sequence set forth in SEQ ID NO: 160,
(15) a light chain CDR1 having an amino acid sequence set forth in SEQ ID NO: 161, a light chain CDR2 having an amino acid sequence set forth in SEQ ID NO: 162, and a light chain CDR3 having an amino acid sequence set forth in SEQ ID NO: 163,
(16) a light chain CDR1 having an amino acid sequence set forth in SEQ ID NO: 173, a light chain CDR2 having an amino acid sequence set forth in SEQ ID NO: 174, and a light chain CDR3 having an amino acid sequence set forth in SEQ ID NO: 175,
(17) a light chain CDR1 having an amino acid sequence set forth in SEQ ID NO: 176, a light chain CDR2 having the amino acid sequence set forth in SEQ ID NO: 177, and a light chain CDR3 having an amino acid sequence set forth in SEQ ID NO: 178, or
(18) a light chain CDR1 having an amino acid sequence set forth in SEQ ID NO: 179, a light chain CDR2 having an amino acid sequence set forth in SEQ ID NO: 180, and a light chain CDR3 having an amino acid sequence set forth in SEQ ID NO: 181.

5: The antibody or the antigen-binding fragment thereof according to any one of above 1 to 4, in which the antibody includes any of the heavy chain variable regions defined in above 3 and any of the light chain variable regions defined in above 4.

6: The antibody or the antigen-binding fragment thereof according to any one of above 1 to 5, in which
the heavy chain variable region includes a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106, and
the light chain variable region includes a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151.

7: The antibody or the antigen-binding fragment thereof according to any one of above 1 to 6, in which the heavy chain variable region includes a framework region 1 having an amino acid sequence set forth in SEQ ID NO: 32, a framework region 2 having an amino acid sequence set forth in SEQ ID NO: 40, a framework region 3 having an amino acid sequence set forth in SEQ ID NO: 46, and a framework region 4 having an amino acid sequence set forth in SEQ ID NO: 54.

8: The antibody or the antigen-binding fragment thereof according to any one of above 1 to 7, in which the light chain variable region includes a framework region 1 having an amino acid sequence set forth in SEQ ID NO: 57, a framework region 2 having an amino acid sequence set forth in SEQ ID NO: 72, a framework region 3 having an amino acid sequence set forth in SEQ ID NO: 79, and a framework region 4 having an amino acid sequence set forth in SEQ ID NO: 86.

9: The antibody or the antigen-binding fragment thereof according to any one of above 1 to 8, in which
the heavy chain variable region has an amino acid sequence selected from the group consisting of SEQ ID NOS: 1 to 6, 8, and 9, and
the light chain variable region has an amino acid sequence selected from the group consisting of SEQ ID NOS: 10, 12 to 15, 17 to 21, and 29 to 31.

10: The antibody or the antigen-binding fragment thereof according to any one of above 1 to 9, in which
the heavy chain variable region has an amino acid sequence set forth in SEQ ID NO: 6, and
the light chain variable region has an amino acid sequence set forth in SEQ ID NO: 21.

11: A composition for use in targeting a HEG1 protein expressed in mesothelioma, the composition comprising the antibody or the antigen-binding fragment thereof.

12: A use of the antibody or the antigen-binding fragment thereof in a manufacture of a composition for use in targeting a HEG1 protein expressed in mesothelioma.

13: A method of targeting a HEG1 protein expressed in mesothelioma in a subject, the method comprising injecting the subject with an effective amount of a composition including the antibody or the antigen-binding fragment thereof.

### BRIEF DESCRIPTION OF DRAWINGS

FIG. 1 illustrates a binding activity of a humanized antibody including a combination of a humanized heavy chain variable region and a humanized light chain variable region to HEG1. Heavy and light chain information of the antibodies used accompanies each bar. Activity has been compared to murine antibodies (parental antibodies) with a murine heavy chain variable region (xiH) and a murine light chain variable region (xiL).

FIG. 2 illustrates the binding activity of the humanized antibody including the combination of the humanized heavy chain variable region and the humanized light chain variable region to HEG1. Heavy and light chain information of the antibodies used accompanies each bar. (−) indicates background in an absence of the antibodies.

FIG. 3 illustrates the binding activity of the humanized antibody including the combination of the humanized heavy chain variable region and the humanized light chain variable region to HEG1. Heavy and light chain information of the antibodies used accompanies each bar.

FIG. 4 illustrates the binding activity of the humanized antibody including the combination of the humanized heavy chain variable region and the humanized light chain variable region to HEG1. Heavy and light chain information of the antibodies used accompanies each bar.

FIG. 5 illustrates the binding activity of the humanized antibody including the combination of the humanized heavy chain variable region and the humanized light chain variable region to HEG1. Heavy and light chain information of the antibodies used accompanies each bar.

FIG. 6 illustrates the binding activity of the humanized antibody including the combination of the humanized heavy chain variable region and the humanized light chain variable region to HEG1. Heavy and light chain information of the antibodies used accompanies each bar.

FIG. 7 illustrates an influence of a signal peptide on a production amount of each humanized antibody.

FIG. 8 is a diagram illustrating a result of SDS-PAGE of each antibody purified from a culture supernatant by expressing each heavy chain and light chain in a CHO cell system.

FIG. 9 illustrates results of Biacore measurement of an affinity of each antibody of purified humanized SKM9-2 to a synthetic sugar epitope in a left column, and results of flow cytometry measurement of the binding of each antibody of purified humanized SKM9-2 to a mesothelioma cell line NCI-H226 in a right column.

### DESCRIPTION OF EMBODIMENTS

### Definitions

In the present specification, the “subject” may be a mammal, and examples thereof include primates such as humans, marmosets, and chimpanzees, experimental animals such as rats, mice, and rabbits, livestock animals such as pigs, cows, horses, sheep, and goats, and pet animals such as dogs and cats, but the “subject” is preferably a human. More preferably, the subject may be a “cancer patient” suffering from or at risk of a tumor or cancer. More preferably, the subject may be a subject suffering from or having the possibility of having mesothelioma. “Patient” means a subject suffering from cancer and is preferably, but not limited to, a human.

In the present specification, the “antibody” means an immunoglobulin, and refers to a protein having a structure in which two heavy chains (H chains) and two light chains (L chains) stabilized by disulfide bonds are associated with each other. The heavy chain consists of a heavy chain variable region VH, heavy chain constant regions CH1, CH2, and CH3, and a hinge region located between CH1 and CH2, and the light chain consists of a light chain variable region VL and a light chain constant region CL. Among them, the variable region fragment (Fv) composed of VH and VL is a region directly involved in antigen binding and giving diversity to the antibody. In addition, an antigen binding region composed of VL, CL, VH, and CH1 is referred to as a Fab region, and a region composed of a hinge region, CH2, and CH3 is referred to as an Fc region.

Among the variable regions, a region that comes into direct contact with an antigen has a particularly large variation, and is called a complementarity-determining region (CDR). A portion having a relatively small variation other than the CDR is referred to as a framework region (FR). Three CDRs exist in each of the variable regions of the light chain and the heavy chain, and are referred to as heavy chain CDRs 1 to 3 and light chain CDRs 1 to 3 in order from the N-terminal side. Each CDR is incorporated into a framework region. The heavy chain variable region of the antibody has, from the N-terminal side to the C-terminal side, a heavy chain framework region 1, a heavy chain CDR1, a heavy chain framework region 2, a heavy chain CDR2, a heavy chain framework region 3, a heavy chain CDR3, and a heavy chain framework region 4 in this order. The light chain variable region of the antibody has, from the N-terminal side to the C-terminal side, a light chain framework region 1, a light chain CDR1, a light chain framework region 2, a light chain CDR2, a light chain framework region 3, a light chain CDR3, and a light chain framework region 4 in this order. The antibody may be a recombinant protein (recombinant antibody) and may be produced in an animal cell, for example, a Chinese hamster ovary cell (CHO cell). The origin of the antibody is not particularly limited, and examples thereof include a non-human animal antibody, a non-human mammal antibody (for example, mouse antibody, rat antibody, camel antibody), and a human antibody. The antibody may also be a chimeric antibody, a humanized antibody, and a fully humanized antibody. The antibody may be a polyclonal antibody or a monoclonal antibody, and is preferably a monoclonal antibody. The “chimeric antibody” is an antibody in which a heavy chain constant region and a light chain constant region of different species are linked to a heavy chain variable region and a light chain variable region, respectively. The humanized antibody means an antibody in which the corresponding position of a human antibody is substituted with an amino acid sequence characteristic of a non-human-derived antibody, and examples thereof include an antibody having the heavy chain CDRs 1 to 3 and the light chain CDRs 1 to 3 of an antibody produced by immunizing a mouse or a rat, in which all other regions including four framework regions (FR) of each of the heavy chain and the light chain are derived from a human antibody. Such antibodies may also be referred to as CDR-grafted antibodies. A “humanized antibody” may also include a human chimeric antibody. A “human chimeric antibody” is an antibody in which the constant region of a non-human-derived antibody is substituted with the constant region of a human antibody in a non-human-derived antibody. In addition, the antibody may be a bispecific antibody. The antibody may be an isolated antibody or a purified antibody. The antibody can be, for example, IgG. The antibody can be, for example, IgG1, IgG2 (For example, IgG2a and IgG2b), IgG3, or IgG4.

The variable regions of immunoglobulin chains generally exhibit the same overall structure, including a relatively conserved framework region (FR) linked by three hypervariable regions (more often referred to as “complementarity determining regions” or CDRs). The CDRs obtained from the two chains of each of the above heavy/light chain pairs are typically juxtaposed by framework regions to form a structure that specifically binds a specific epitope on a target protein (for example, PCSK9). From the N-terminal side to the C-terminal side, any naturally occurring light and heavy chain variable regions typically conform to the following sequence of these elements. FR1, CDR1, FR2, CDR2, FR3, CDR3 and FR4. Numbering systems have been devised to assign numbers to the amino acids occupying positions in each of these domains. This numbering system is defined in “Kabat Sequences of Proteins of Immunological Interest (1987 and 1991, NIH, Bethesda, MD)” or “Chothia & Lesk, 1987, J. Mol. Biol. 196:901-917; Chothia et al., 1989, Nature 342:878-883”.

In the present specification, the term “antigen-binding fragment” means a part of an antibody that maintains binding to an antigen. The antigen-binding fragment may include a heavy chain variable region or a light chain variable region, or both, of an antibody of the present disclosure. The antigen-binding fragment may be chimerized or humanized. Examples of the antigen-binding fragment include Fab, Fab′, F(ab′)₂, and Fv. In addition, the antibody-binding fragment may also include recombinantly produced conjugates or functional equivalents (for example, some other antibodies having the form of scFv (single chain Fv), diabody, scDb, tandem scFv, leucine zipper type, sc(Fv)₂ (single chain (Fv)₂), or the like). The antigen-binding fragment of such an antibody is not particularly limited, but can be obtained, for example, by treating the antibody with an enzyme. For example, the antibody can be digested with papain to obtain yield Fab. Alternatively, the antibody can be digested with pepsin to yield F(ab′)₂, which can be further reduced to yield Fab′. In the present specification, the antigen-binding fragment of such antibodies can be used. In scFv, VL and VH can be linked with an artificial polypeptide linker to maintain the same antigen specificity as the original antibody. VL and VH may be linked from the N-terminal side in the order of VH and VL or VL and VH. The linker may have a length of the order of 10 to 25 amino acids. The linker may be rich in glycine and may contain an amino acid such as serine or threonine for the purpose of enhancing water solubility.

In the present specification, the “HEG1 protein” is a protein expressed in the membrane of mesothelioma cells (WO 2017/141604). According to WO 2017/141604, it is considered that the HEG1 protein is subjected to sugar modification on the membrane of mesothelioma cells. The sugar chain modification includes O-type sugar chain modification. The sugar chain modification is sialylated. The sugar chain modification may include α2,3 sialylation. The sugar modification is considered to be mesothelioma-specific. Therefore, according to WO 2017/141604, mesothelioma cells can be detected by an antibody that binds to a HEG1 protein having this sugar modification. Examples of the human HEG1 proteins include proteins having a nucleic acid sequence registered as NM_020733.1 at the National Center for Biotechnology Information (NCBI) and the amino acid sequence encoded thereby. From the result of gene ontology analysis, it is predicted that in the HEG1 protein, a signal peptide moiety will be a domain corresponding to positions 1 to 29 of the amino acid sequence, an extracellular domain will be a domain corresponding to positions 30 to 1248 of the amino acid sequence, a transmembrane domain will be a domain corresponding to positions 1249 to 1269 of the amino acid sequence, and an intracellular domain will be a domain corresponding to positions 1270 to 1381 of the amino acid sequence. For example, the HEG1 protein may also include a protein having an amino acid sequence 90% or more, 95% or more, 98% or more, or 99% or more homologous to the above-described amino acid sequence. For example, the HEG1 protein may contain one or more amino acid substitutions, insertions, additions and/or deletions in the amino acid sequence represented by the amino acid sequence.

In the present specification, the amino acid sequence is represented by a one-letter code. That is, A represents alanine, R represents arginine, N represents asparagine, D represents aspartic acid, C represents cysteine, Q represents glutamine, E represents glutamic acid, G represents glycine, H represents histidine, I represents isoleucine, L represents leucine, K represents lysine, M represents methionine, F represents phenylalanine, P represents proline, S represents serine, T represents threonine, W represents tryptophan, Y represents tyrosine, and V represents valine.

In the present specification, “mesothelioma” means a tumor derived from mesothelial cells. As the mesothelioma, pleural mesothelioma, peritoneal mesothelioma, pericardial mesothelioma, and testicular sheath mesothelioma are known from the generation site thereof. In the present specification, mesothelioma means benign mesothelioma and/or malignant mesothelioma. Mesothelioma is roughly classified into epithelial mesothelioma, sarcomatous mesothelioma, biphasic mesothelioma, and other mesotheliomas (fibrogenic type, or the like) from the tissue type. In certain aspects, the mesothelioma can be malignant mesothelioma.

### <Humanized Antibody of Present Disclosure>

In the present disclosure, the antibody is an antibody that binds to a HEG1 protein. The HEG1 protein may be the HEG1 protein expressed in mesothelioma cells (for example, ACC-MESO4 cell line) (WO 2017/141604). The HEG1 protein expressed in mesothelioma cells may include mesothelioma-specific sugar chain modifications. Since the sugar chain modification is decomposed by α2,3-neuraminidase treatment, it includes α2,3-sialylation (WO 2017/141604). In addition, the sugar chain modification may be an O-type sugar chain modification because it is not degraded by N-glycanase (PNGase F) (WO 2017/141604).

In certain preferred aspects, the antibodies of the present disclosure bind to the HEG1 protein expressed in the mesothelioma cells. In certain preferred aspects, the antibody binds to the HEG1 protein in a manner dependent on an O-type sugar chain modification containing α2,3 sialic acid. That is, the antibody can partially or completely lose binding to the HEG1 protein by α2,3 neuraminidase treatment. The antibody can also partially or completely lose binding to HEG1 protein by proteinase K treatment.

According to the present disclosure, the humanized antibody that binds to a HEG1 (particularly human HEG1) protein is provided. In certain preferred aspects, the antibodies of the present disclosure bind to the HEG1 protein expressed in the mesothelioma cells. In certain preferred aspects, the antibody binds to the HEG1 protein in a manner dependent on an O-type sugar chain modification containing α2,3 sialic acid. That is, the antibody can partially or completely lose binding to the HEG1 protein by α2,3 neuraminidase treatment. The antibody can also partially or completely lose binding to HEG1 protein by proteinase K treatment.

In certain aspects, the antibody of the present disclosure is an antibody that binds to a HEG1 protein expressed in mesothelioma cells (for example, ACC-MESO4 cell line). Such an antibody can be obtained by a conventional method, for example, as described in WO 2017/141604. Specifically, for example, the antibody binds to a HEG1 protein expressed in mesothelioma cells (for example, ACC-MESO4 cell line), but in certain aspects, the binding can be eliminated or reduced by protease K treatment of the HEG1 protein. For example, the antibody binds to the HEG1 protein expressed in mesothelioma cells (for example, ACC-MESO4 cell line), but in certain aspects, the binding can be eliminated or reduced by α2,3 neuraminidase treatment. In certain aspects, the binding is not eliminated by treatment with one or more or any selected from the group consisting of β-N-acetylglucosaminidase, N-glycanase (PNGaseF), lysozyme, and hyaluronidase. The processing is performed under conditions suitable for the individual processing. In certain aspects, the antibody can be a human antibody. The human antibody can be made by immunizing a non-human mammal (for example, mouse) incorporating human IgG loci with an immunogen. In certain aspects, a human heavy chain variable region is inserted upstream of the heavy chain constant region of mouse IgG, and a mouse having a human light chain variable region inserted upstream of the light chain constant region of mouse IgG is immunized with an immunogen to obtain an antibody as a human chimeric antibody, and the constant region can be replaced with a corresponding human constant region (see, for example, WO 2002/066630 A and WO 2011/004192 A.). The humanized antibody can be generated by removing the six CDRs of a human antibody and replacing them with the corresponding CDRs (six CDRs) of the resulting antibody.

In certain aspects, an antibody or antigen-binding fragment thereof of the present disclosure is a humanized antibody that binds to a partial peptide of a HEG1 (particularly human HEG1) protein. A partial peptide can be, for example, an antibody which binds to a peptide having an amino acid sequence set forth in SEQ ID NO: 182 (SKSPSLVSLPT). The partial peptide can be, for example, a peptide produced by mesothelioma cells (for example, ACC-MESO4 cell line). The peptide can be obtained as a fusion protein by being linked to the N-terminal side of a protein (SEQ ID NO: 183; hereinafter, referred to as “SLURPgpi”; the signal sequence is shown in SEQ ID NO: 184) in which a GPI anchor signal is linked to the N-terminal of human SLURP1, for example, and can be used for evaluation of binding to an antibody. In certain preferred aspects, in the peptide having an amino acid sequence set forth in SEQ ID NO: 182 (SKSPSLVSLPT), either or both of the first serine and the eighth serine have a sugar chain modification. In certain preferred aspects, the sugar chain modification can be a 2,3-sialyl T antigen (2,3-Sialyl T), or a disialyl T antigen (DiSialyl T). In a most preferred aspect, there is provided an antibody that is for an antigen having an amino acid sequence set forth in SEQ ID NO: 182 (SKSPSLVSLPT), in which either or both of the first serine and the eighth serine have a sugar chain modification, and the sugar chain modification is 2,3-sialyl T antigen (2,3-Sialyl T) or disialyl T antigen (DiSialyl T).

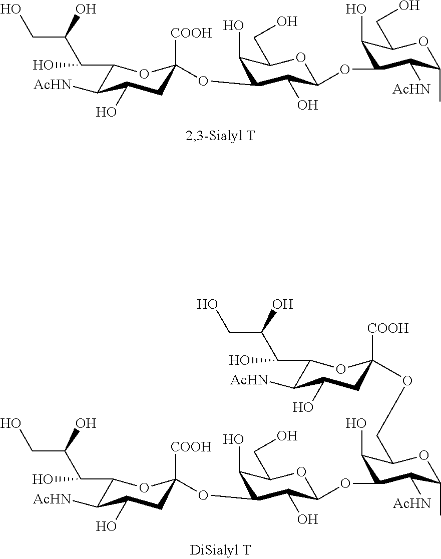

In certain aspects, a humanized antibody of the present disclosure may include a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 62, a light chain CDR2 having an amino acid sequence set forth in SEQ ID NO: 75, and a light chain CDR3 having an amino acid sequence set forth in SEQ ID NO: 82.

In certain aspects, a humanized antibody of the present disclosure may include a heavy chain variable region including a heavy chain CDR1 having the amino acid sequence set forth in SEQ ID NO: 37, a heavy chain CDR2 having the amino acid sequence set forth in SEQ ID NO: 43, and a heavy chain CDR3 having the amino acid sequence set forth in SEQ ID NO: 51, and a light chain variable region including a light chain CDR1 having the amino acid sequence set forth in SEQ ID NO: 63, a light chain CDR2 having the amino acid sequence set forth in SEQ ID NO: 75, and a light chain CDR3 having the amino acid sequence set forth in SEQ ID NO: 82.

In certain aspects, the humanized antibody of the present disclosure may include a heavy chain variable region having any of the following heavy chain CDRs 1 to 3 sets (1A) to (8A):
(1A) a heavy chain CDR1 having the amino acid sequence set forth in SEQ ID NO: 89, a heavy chain CDR2 having the amino acid sequence set forth in SEQ ID NO: 90, and a heavy chain CDR3 having the amino acid sequence set forth in SEQ ID NO: 91;
(2A) a heavy chain CDR1 having the amino acid sequence set forth in SEQ ID NO: 92, a heavy chain CDR2 having the amino acid sequence set forth in SEQ ID NO: 93, and a heavy chain CDR3 having the amino acid sequence set forth in SEQ ID NO: 94;
(3A) a heavy chain CDR1 having the amino acid sequence set forth in SEQ ID NO: 95, a heavy chain CDR2 having the amino acid sequence set forth in SEQ ID NO: 96, and a heavy chain CDR3 having the amino acid sequence set forth in SEQ ID NO: 97;
(4A) a heavy chain CDR1 having the amino acid sequence set forth in SEQ ID NO: 98, a heavy chain CDR2 having the amino acid sequence set forth in SEQ ID NO: 99, and a heavy chain CDR3 having the amino acid sequence set forth in SEQ ID NO: 100;
(5A) a heavy chain CDR1 having the amino acid sequence set forth in SEQ ID NO: 101, a heavy chain CDR2 having the amino acid sequence set forth in SEQ ID NO: 102, and a heavy chain CDR3 having the amino acid sequence set forth in SEQ ID NO: 103;
(6A) a heavy chain CDR1 having the amino acid sequence set forth in SEQ ID NO: 104, a heavy chain CDR2 having the amino acid sequence set forth in SEQ ID NO: 105, and a heavy chain CDR3 having the amino acid sequence set forth in SEQ ID NO: 106;
(7A) a heavy chain CDR1 having the amino acid sequence set forth in SEQ ID NO: 110, a heavy chain CDR2 having the amino acid sequence set forth in SEQ ID NO: 111, and a heavy chain CDR3 having the amino acid sequence set forth in SEQ ID NO: 112; and
(8A) a heavy chain CDR1 having the amino acid sequence set forth in SEQ ID NO: 113, a heavy chain CDR2 having the amino acid sequence set forth in SEQ ID NO: 114, and a heavy chain CDR3 having the amino acid sequence set forth in SEQ ID NO: 115.

The humanized antibody of the present disclosure may include a heavy chain variable region including the heavy chain CDRs 1 to 3 of any of (1A) to (8A) above.

In certain aspects, the humanized antibody of the present disclosure may include a light chain variable region having any of the following light chain CDRs 1 to 3 sets (1B) to (18B):
(1B) a light chain CDR1 having the amino acid sequence set forth in SEQ ID NO: 116, a light chain CDR2 having the amino acid sequence set forth in SEQ ID NO: 117, and a light chain CDR3 having the amino acid sequence set forth in SEQ ID NO: 118;
(2B) a light chain CDR1 having the amino acid sequence set forth in SEQ ID NO: 119, a light chain CDR2 having the amino acid sequence set forth in SEQ ID NO: 120, forth in SEQ ID NO: 121;
(3B) a light chain CDR1 having the amino acid sequence set forth in SEQ ID NO: 122, a light chain CDR2 having the amino acid sequence set forth in SEQ ID NO: 123, and a light chain CDR3 having the amino acid sequence set forth in SEQ ID NO: 124;
(4B) a light chain CDR1 having the amino acid sequence set forth in SEQ ID NO: 128, a light chain CDR2 having the amino acid sequence set forth in SEQ ID NO: 129, and a light chain CDR3 having the amino acid sequence set forth in SEQ ID NO: 130;
(5B) a light chain CDR1 having the amino acid sequence set forth in SEQ ID NO: 131, a light chain CDR2 having the amino acid sequence set forth in SEQ ID NO: 132, and a light chain CDR3 having the amino acid sequence set forth in SEQ ID NO: 133;
(6B) a light chain CDR1 having the amino acid sequence set forth in SEQ ID NO: 134, a light chain CDR2 having the amino acid sequence set forth in SEQ ID NO: 135, and a light chain CDR3 having the amino acid sequence set forth in SEQ ID NO: 136;
(7B) a light chain CDR1 having the amino acid sequence set forth in SEQ ID NO: 137, a light chain CDR2 having the amino acid sequence set forth in SEQ ID NO: 138, forth in SEQ ID NO: 139;
(8B) a light chain CDR1 having the amino acid sequence set forth in SEQ ID NO: 140, a light chain CDR2 having the amino acid sequence set forth in SEQ ID NO: 141, and a light chain CDR3 having the amino acid sequence set forth in SEQ ID NO: 142;
(9B) a light chain CDR1 having the amino acid sequence set forth in SEQ ID NO: 143, a light chain CDR2 having the amino acid sequence set forth in SEQ ID NO: 144, and a light chain CDR3 having the amino acid sequence set forth in SEQ ID NO: 145;
(10B) a light chain CDR1 having the amino acid sequence set forth in SEQ ID NO: 146, a light chain CDR2 having the amino acid sequence set forth in SEQ ID NO: 147, and a light chain CDR3 having the amino acid sequence set forth in SEQ ID NO: 148;
(11B) a light chain CDR1 having the amino acid sequence set forth in SEQ ID NO: 149, a light chain CDR2 having the amino acid sequence set forth in SEQ ID NO: 150, and a light chain CDR3 having the amino acid sequence set forth in SEQ ID NO: 151;
(12B) a light chain CDR1 having the amino acid sequence set forth in SEQ ID NO: 152, a light chain CDR2 having the amino acid sequence set forth in SEQ ID NO: 153, forth in SEQ ID NO: 154;
(13B) a light chain CDR1 having the amino acid sequence set forth in SEQ ID NO: 155, a light chain CDR2 having the amino acid sequence set forth in SEQ ID NO: 156, and a light chain CDR3 having the amino acid sequence set forth in SEQ ID NO: 157;
(14B) a light chain CDR1 having the amino acid sequence set forth in SEQ ID NO: 158, a light chain CDR2 having the amino acid sequence set forth in SEQ ID NO: 159, and a light chain CDR3 having the amino acid sequence set forth in SEQ ID NO: 160;
(15B) a light chain CDR1 having the amino acid sequence set forth in SEQ ID NO: 161, a light chain CDR2 having the amino acid sequence set forth in SEQ ID NO: 162, and a light chain CDR3 having the amino acid sequence set forth in SEQ ID NO: 163;
(16B) a light chain CDR1 having the amino acid sequence set forth in SEQ ID NO: 173, a light chain CDR2 having the amino acid sequence set forth in SEQ ID NO: 174, and a light chain CDR3 having the amino acid sequence set forth in SEQ ID NO: 175;
(17B) a light chain CDR1 having the amino acid sequence set forth in SEQ ID NO: 176, a light chain CDR2 having the amino acid sequence set forth in SEQ ID NO: 177, forth in SEQ ID NO: 178; and
(18B) a light chain CDR1 having the amino acid sequence set forth in SEQ ID NO: 179, a light chain CDR2 having the amino acid sequence set forth in SEQ ID NO: 180, and a light chain CDR3 having the amino acid sequence set forth in SEQ ID NO: 181.

The humanized antibody of the present disclosure may include a light chain variable region including the light chain CDRs 1 to 3 of any of (1B) to (18B) above.

In certain aspects, an antibody of the present disclosure may include
a heavy chain variable region including a heavy chain CDR1 having the amino acid sequence set forth in SEQ ID NO: 37, a heavy chain CDR2 having the amino acid sequence set forth in SEQ ID NO: 43, and a heavy chain CDR3 having the amino acid sequence set forth in SEQ ID NO: 51, and
a light chain variable region including the set of light chain CDRs 1 to 3 of any of (1B) to (18B) above.

In certain aspects, an antibody of the present disclosure may include
a heavy chain variable region including the set of heavy chain CDRs 1 to 3 of any of (1A) to (8A) above; and
a light chain variable region including a light chain CDR1 having the amino acid sequence set forth in SEQ ID NO: 62, a light chain CDR2 having the amino acid sequence set forth in SEQ ID NO: 75, and a light chain CDR3 having the amino acid sequence set forth in SEQ ID NO: 82. Here, the light chain CDR1 may preferably have the amino acid sequence set forth in SEQ ID NO: 63.

In certain aspects, an antibody of the present disclosure may include:
a heavy chain variable region including the set of heavy chain CDRs 1 to 3 of any of (1A) to (8A) above; and
a light chain variable region including the set of light chain CDRs 1 to 3 of any of (1B) to (18B) above.

In certain aspects, an antibody of the present disclosure may include:
a heavy chain variable region including the set of heavy chain CDRs 1 to 3 of (1A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (2A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (3A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (4A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (5A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) and (10B) above (in particular any of (4B), (7B), (10B) or (14B) to (16B)). In certain preferred aspects, the light chain variable region can be (10B) above.

In certain aspects, an antibody of the present disclosure may include:
a heavy chain variable region including the set of heavy chain CDRs 1 to 3 of (6A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) and (10B) above (in particular any of (4B), (7B), (10B) or (14B) to (16B)). In certain preferred aspects, the light chain variable region can be (10B) above.

In certain aspects, an antibody of the present disclosure may include:
a heavy chain variable region including the set of heavy chain CDRs 1 to 3 of (7A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (8A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain preferred aspects, in the antibody of the present disclosure, the variable region of the antibody to which the heavy chain CDRs 1 to 3 and the light chain CDRs 1 to 3 are grafted may be a variable region of a human IgG1 antibody. In certain aspects, in the antibody of the present disclosure, the variable region of the antibody to which the heavy chain CDRs 1 to 3 and the light chain CDRs 1 to 3 are grafted may be a variable region of a human IgG2 antibody. In certain aspects, in the antibody of the present disclosure, the variable region of the antibody to which the heavy chain CDRs 1 to 3 and the light chain CDRs 1 to 3 are grafted may be a variable region of a human IgG3 antibody. In certain aspects, in the antibody of the present disclosure, the variable region of the antibody to which the heavy chain CDRs 1 to 3 and the light chain CDRs 1 to 3 are grafted may be a variable region of a human IgG4 antibody.

In certain preferred aspects, in the antibody of the present disclosure, the Fc region (that is, heavy chain constant regions 2 and/or 3) may be the Fc region of the human IgG1 antibody. In certain preferred aspects, in the antibody of the present disclosure, the Fc region (that is, heavy chain constant regions 2 and/or 3) may be the Fc region of the human IgG2 antibody. In certain preferred aspects, in the antibody of the present disclosure, the Fc region (that is, heavy chain constant regions 2 and/or 3) may be the Fc region of the human IgG3 antibody. In certain preferred aspects, in the antibody of the present disclosure, the Fc region (that is, heavy chain constant regions 2 and/or 3) may be the Fc region of the human IgG4 antibody.

In certain aspects, in the antibody of the present disclosure, the heavy chain variable region may include
a framework region 1 having the amino acid sequence set forth in SEQ ID NO: 32, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54.

In certain preferred aspects, in the antibody of the present disclosure, the heavy chain variable region includes a framework region 1 having the amino acid sequence set forth in SEQ ID NO: 33, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54. Wherein, in SEQ ID NO: 46, X1 may be A, X3 may be T, X5 may be E, X6 may be R, and X7 may be T, and in SEQ ID NO: 54, X1 may be V, X3 may be T, X4 may be Q or E, and X5 may be P.

In certain aspects, in the antibody of the present disclosure, the heavy chain variable region includes a framework region 1 having the amino acid sequence set forth in SEQ ID NO: 33, a framework region 2 having the amino acid sequence set forth in SEQ ID NO: 42, a framework region 3 having the amino acid sequence set forth in SEQ ID NO: 48, and a framework region 4 having the amino acid sequence set forth in SEQ ID NO: 59.

In certain aspects, in the antibody of the present disclosure, the light chain variable region may include a framework region 1 having the amino acid sequence set forth in SEQ ID NO: 57, a framework region 2 having the amino acid sequence set forth in SEQ ID NO: 72, a framework region 3 having the amino acid sequence set forth in SEQ ID NO: 79, and a framework region 4 having the amino acid sequence set forth in SEQ ID NO: 86.

In certain aspects, in the antibody of the present disclosure, the light chain variable region may include a framework region 1 having the amino acid sequence set forth in SEQ ID NO: 61, a framework region 2 having the amino acid sequence set forth in SEQ ID NO: 74, a framework region 3 having the amino acid sequence set forth in SEQ ID NO: 81, and a framework region 4 having the amino acid sequence set forth in SEQ ID NO: 88.

In certain aspects, an antibody of the present disclosure may include one heavy chain variable region selected from the group consisting of SEQ ID NOs: 1 to 6, 8, and 9 and one light chain variable region selected from the group consisting of SEQ ID NOs: 10, 12 to 15, 17 to 21, 23 to 25, and 29 to 31. In certain aspects, the antibody of the present disclosure may include one heavy chain variable region selected from the group consisting of SEQ ID NOs: 2, 4 to 6, 8, and 9 and one light chain variable region selected from the group consisting of SEQ ID NOS: 14, 15, 17 to 21, 23 to 25, and 29 to 31. In certain preferred aspects, the antibody of the present disclosure may include a heavy chain variable region set forth in SEQ ID NO: 6 and a light chain variable region set forth in SEQ ID NO: 21.

The antibody or antigen-binding fragment thereof of the present disclosure may include an antibody or antigen-binding fragment thereof having a mutation selected from the group consisting of insertion, deletion, addition and substitution of one to several amino acids. In certain embodiments, provided is an antibody or an antigen-binding fragment thereof including at least one CDR, at least two, at least three, or more CDRs substantially identical to at least one CDR, at least two, at least three, or more CDRs in the antibody or the antigen-binding fragment thereof of the present disclosure. In another embodiment, an antibody having at least two, three, four, five, or six CDRs substantially identical to at least two, three, four, five, or six CDRs in or derived from the antibody or the antigen-binding fragment thereof of the present disclosure is included. In certain embodiments, at least one, two, or three CDRs in the antibody or the antigen-binding fragment thereof of the present disclosure and at least one, two, three, four, five, or six CDRs that are at least about 85%, 86%, 87%, 88%, 89%, 90%, 95%, 96%, 97%, 98%, or 99% identical are included. In certain embodiments, at least one, two, three, four, five, or six CDRs include at least one insertion, deletion, addition, or substitution in the antibody or the antigen-binding fragment thereof of the present disclosure or in at least one, two, three, four, five, or six CDRs derived from the antibody or the antigen-binding fragment thereof of the present disclosure. The antibody or the antigen-binding fragment thereof of the present disclosure may include an antibody or antigen-binding fragment thereof having an amino acid sequence identity of 80% or more, 85% or more, 90% or more, or 95% or more and having antigenic specificity of the antibody. The antibody or the antigen-binding fragment thereof of the present disclosure may include, for example, an antibody or an antigen-binding fragment thereof having 80% or more, 85% or more, 90% or more, or 95% or more amino acid sequence identity with the antibody disclosed above within a framework region and having antigen-specificity of the antibody. The antibody or the antigen-binding fragment thereof of the present disclosure may include, for example, an antibody or an antigen-binding fragment thereof having a mutation selected from the group consisting of insertion, deletion, addition and substitution of one to several amino acids in the antibody disclosed above within a framework region.

In certain preferred aspects, the humanized IgG antibody can be an antibody that binds to a peptide having the amino acid sequence set forth in SEQ ID NO: 182 (SKSPSLVSLPT). The partial peptide can be, for example, a peptide produced by mesothelioma cells (for example, ACC-MESO4 cell line). The peptide can be obtained as a fusion protein by being linked to the N-terminal side of a protein in which a GPI anchor signal is linked to the N-terminal of human SLURP1, for example, and can be used for evaluation of binding to an antibody. In certain preferred aspects, the human antibody can be any of the human antibodies described above.

In certain preferred aspects, the humanized IgG antibody can be any of the humanized antibodies described above. In certain preferred aspects, the antibody of the present disclosure may include a heavy chain variable region set forth in SEQ ID NO: 6 and a light chain variable region set forth in SEQ ID NO: 21.

Non-limiting examples of the heavy chain constant region of the IgG antibody include the heavy chain constant regions of IgG1, IgG2, IgG3, IgG4, and IgG4PE, and the heavy chain constant regions of IgG1, IgG2, IgG4, and IgG4PE are preferred, and the heavy chain constant regions of IgG1 and IgG4PE are more preferably, and the heavy chain constant region of IgG4PE can be further preferably used. The hinge region of the IgG1 may include, for example, the amino acid sequence of SEQ ID NO: 185. The hinge region of the IgG4 may, for example, have the amino acid sequence of SEQ ID NO: 186, and the hinge region of the IgG4PE may, for example, have the amino acid sequence of SEQ ID NO: 187. The CH2 region of the IgG1 may include, for example, the amino acid sequence of SEQ ID NO: 188. The CH2 regions of IgG4 and IgG4PE may, for example, have the amino acid sequence of SEQ ID NO: 189.

Non-limiting examples of light chain constant regions of IgG antibodies include light chain constant regions of kappa or lambda chains, for example, light chain constant regions of kappa chains.

The humanized antibody of the present disclosure may or may not have internalizing activity for therapeutic and diagnostic purposes.

### <Method for Treating Mesothelioma in Subject>

According to the present disclosure, a method for treating mesothelioma in a subject is provided. According to the present disclosure, a method includes injecting a subject with an effective amount of an antibody of the present disclosure. The subject may be a subject with mesothelioma. The subject may be a subject diagnosed as having mesothelioma.

The injection may be performed by parenteral injection (for example, intraperitoneal injection, intrapleural injection, intratumoral injection, intravenous injection, or the like). The injected dose can be appropriately determined by a physician in consideration of the condition, weight, sex, and age of the subject. The injection may be a single injection or multiple injections.

According to the present disclosure, the antibody of the present disclosure is provided for use in the above method.

In certain aspects, the antibody of the present disclosure may have antibody-dependent cellular cytotoxicity (ADCC) activity and/or complement-dependent cytotoxicity (CDC) activity. ADCC activity means an activity in which, when the antibody of the present invention binds to a cell surface antigen of a target cell, an Fcγ receptor-bearing cell (effector cell) binds to an Fc portion thereof via an Fcγ receptor and damages the target cell.

ADCC activity can be found by mixing a target cell expressing HEG1 (for example, mesothelioma cells), an effector cell, and an antibody of the present invention, and measuring the degree of ADCC. As the effector cells, for example, mouse splenocytes or monocyte nuclei separated from human peripheral blood or bone marrow can be used. As the target cell, for example, a mesothelioma cell expressing HEG1 can be used. The target cell is previously labeled with 51 Cr or the like, the antibody of the present invention is added thereto and incubated, and then effector cells are added to the target cells at an appropriate ratio and incubated. After the incubation, the supernatant is collected, and the label in the supernatant is counted, whereby the measurement can be performed.

CDC activity means cytotoxic activity due to the complement system. The CDC activity can be measured using complement in place of the effector cell in the ADCC activity test.

In certain aspects, the antibody of the present disclosure may have a tumor growth inhibitory effect in itself. The tumor growth inhibitory activity can be measured utilizing tumor model animals. For example, a tumor is implanted subcutaneously in a mouse, and the antibody of the present invention is injected. By comparing the volume of tumor tissue between the non-injection group and the injection group, the tumor growth inhibitory effect can be measured. The tumor growth inhibitory activity of the present invention may be produced as a result of inhibiting proliferation of individual cells or as a result of inducing cell death.

In certain aspects, the antibody of the present disclosure may be linked via a linker to a cytotoxic agent, particularly a chemotherapeutic agent. Therefore, according to the present disclosure, an antibody-drug conjugate including an antibody of the present disclosure and a cytotoxic agent, in which the antibody and the cytotoxic agent are linked via a linker may be provided. Examples of the cytotoxic agent include chemotherapeutic agents (anti-cancer agents such as, for example, commercially available anti-cancer agents, for example, auristatins (auristatin E, auristatin F phenylenediamine (AFP), monomethylauristatin E, monomethylauristatin F and derivatives thereof), maytansinoids DM1 and DM4 and derivatives thereof), camptothecin (SN-38, irinotecan, roottecan, DB 67, BMP 1350, ST 1481, CKD 602, topotecan and exatecan, and derivatives thereof), DNA minor groove binders (enediyne, lexitropsin, duocarmycin, and derivatives thereof), taxanes (paclitaxel and docetaxel and derivatives thereof), polyketides (discodermolide and derivatives thereof), anthraquinones (mitoxantrone and derivatives thereof), benzodiazepines (pyrrolobenzodiazepines, indolinobenzodiazepines, and oxazolidinobenzodiazepines and derivatives thereof), vinca alkaloids (vincristine, vinblastine, vindesine, and vinorelbine and derivatives thereof), doxorubicines (doxorubicin, morpholino-doxorubicin, and cyanomorpholino-doxorubicin and derivatives thereof), cardiac glycosides (digitoxin and derivatives thereof), calicheamicin, epothilone, cryptophycin, semadotin, semadotin, rhizoxin, netropsin, combretastatin, eleutherobin, etoposide, T 67 (turarik), and nocodazole), and toxins (for example, diphtheria toxin A, pseudomonas endotoxin, ricin, saporin, and the like), and can be used as the cytotoxic agent in the ADC of the present invention. As the cytotoxic agent, a pharmaceutically acceptable salt, solvate (for example, hydrates), ester, or prodrug of the cytotoxic agent may be used.

As the linker, a non-cleavable linker or a cleavable linker (including, for example, valine-citrulline dipeptide) can be used.

According to the present disclosure, there is provided a composition including the antibody of the present disclosure. According to the present disclosure, a pharmaceutical composition including the antibody of the present disclosure is disclosed. The pharmaceutical composition of the present disclosure may be used to treat the mesothelioma. According to the present disclosure, there is also provided a composition including an antibody-drug conjugate of the present disclosure. According to the present disclosure, the pharmaceutical composition including an antibody-drug conjugate of the present disclosure is disclosed. The pharmaceutical composition of the present disclosure may be used to treat the mesothelioma.

The pharmaceutical composition of the present disclosure may further include pharmaceutically acceptable carriers, excipients, and/or additives. Examples of carriers and additives include, but are not limited to, water, saline, phosphate buffer, dextrose, glycerol, ethanol, and other pharmaceutically acceptable organic solvents, collagen, polyvinyl alcohol, polyvinylpyrrolidone, carboxyvinyl polymer, sodium carboxymethylcellulose, sodium polyacrylate, sodium alginate, water-soluble dextran, carboxymethyl starch, pectin, methylcellulose, ethylcellulose, xanthan gum, gum arabic, casein, agar, polyethylene glycol, diglycerin, glycerin, propylene glycol, petrolatum, paraffin, stearyl alcohol, stearic acid, human serum albumin, mannitol, sorbitol, lactose, surfactants, and the like.

The pharmaceutical composition of the present invention can be in various forms, for example, a liquid preparation (for example, an injection preparation). A preferred aspect is an injection preparation or an infusion preparation, and is preferably administered parenterally (for example, intravenous, transdermal, subcutaneous, intradermal, intraperitoneal, intrapleural, intramuscular, or intratumoral).

### EXAMPLES

The present invention will be described more specifically with reference to the following examples, but the scope of the present invention is not limited by these examples.

### Production Example 1: Production of Humanized Antibody

The humanized antibody was designed to have an amino acid sequence of various variable regions to which a signal sequence derived from a mouse antibody was added and an amino acid sequence of a constant region of human IgG1 based on a conventional method. The nucleotide sequence encoding the designed humanized antibody was converted with consideration to be a codon usage suitable for expression in Chinese hamster ovary (CHO) cells. A Kozak sequence was added to the base sequence obtained by conversion and the start codon site of the signal sequence. A plasmid for expressing mammalian cells (pcDNA 3.1) having a stop codon added to the C-terminal side of the constant region and further having a restriction enzyme site added to the upstream of the Kozak sequence and the downstream of the stop codon was each subjected to restriction enzyme treatment and ligated, and the above-described sequence obtained was incorporated into the plasmid. Each of the obtained H chain expression plasmids and each of the L chain expression plasmids were expressed in ExpiCHO Expression System (ThermoFisher Scientific) or RK 13 cells so as to form a combination of one H chain and one L chain, and recovered from the culture supernatant with HiTrap protein G (1 mL) (Cytiba), eluted with 0.1 M glycine buffer (pH 2.8), and purified by dialysis against PBS.

The humanized antibody was obtained by the method described above. The antibody activity of each obtained antibody was measured by ELISA using 7.62 EGF as an antigen. The 7.62 EGF is a secreted protein in which an epitope region of an antibody is linked to the N-terminal side of the EGF domain region of HEG1 and a His tag is linked to the C-terminal side, and when produced in 293 H cells, sialylated sugar chain-modified EGF is recognized by the antibody.

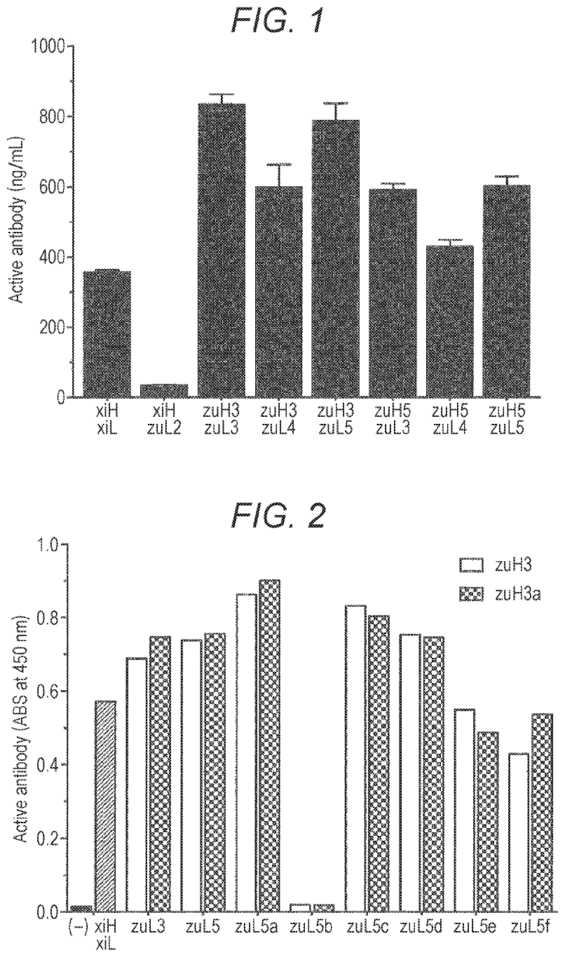

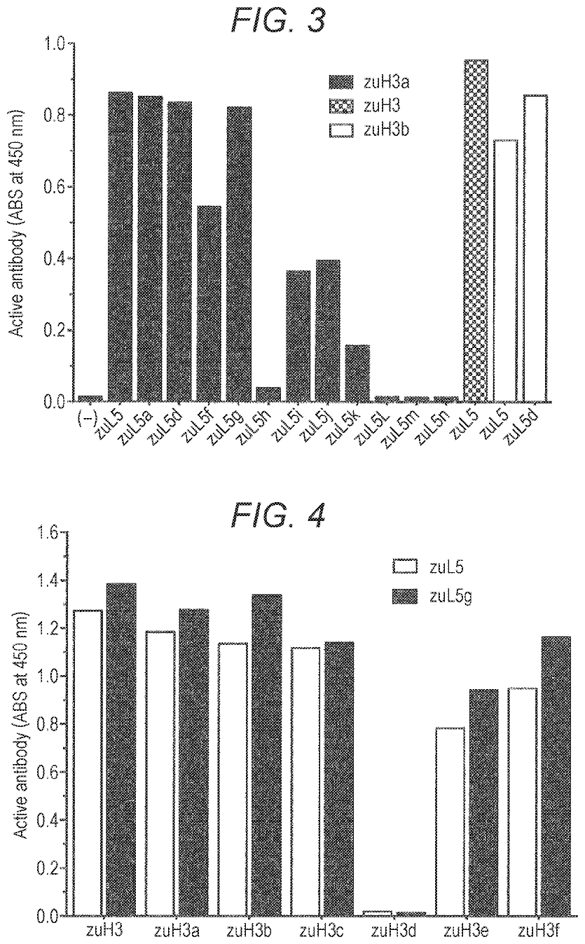

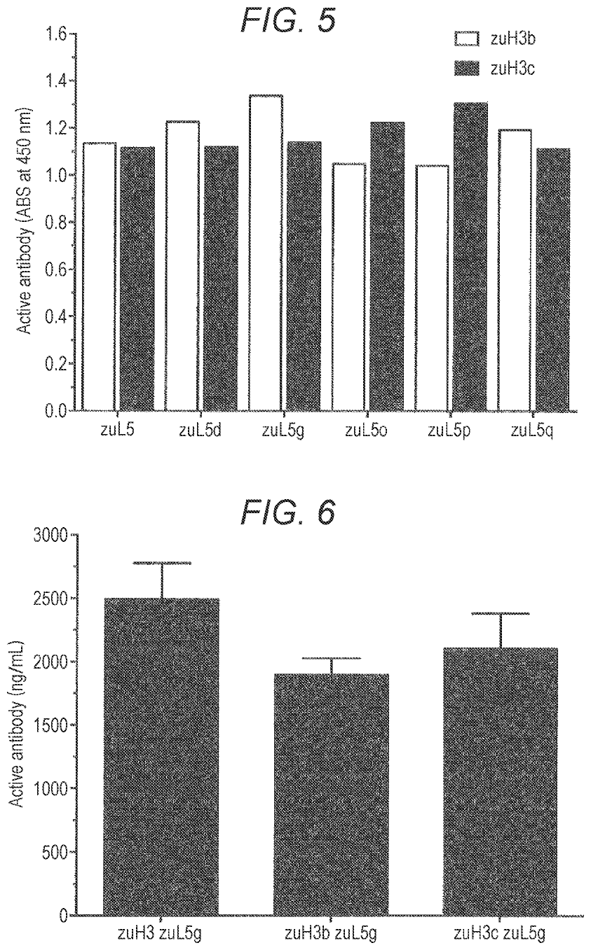

The 7.62 EGF purified from the culture supernatant of homeostatic expressing cells was adsorbed to an ELISA plate and blocked with 1% BSA. A culture supernatant of RK 13 cells into which a humanized antibody gene had been introduced was added to the plate, and the mixture was reacted at room temperature for 3 hours. After washing with 20 mM Tris buffered saline containing 0.1% Tween 20, a secondary antibody (horseradish peroxidase-labeled goat anti-human IgG, Fc gamma fragment specific) was added thereto, and the mixture was reacted for 90 minutes. After washing with 20 mM Tris buffered saline containing 0.1% Tween 20, 1-Step Ultra TMB-ELISA Substrate Solution (Thermo Fisher Scientific) was added. The reaction was stopped with 2 M sulfuric acid, and the absorbance at 450 nm was measured. The results are illustrated in FIGS. 1 to 6. The antigen binding activity was observed with the antibodies containing the combinations of the amino acid sequence shown in Table 1 as a heavy chain and the amino acid sequence shown in Table 2 as a light chain.

| Clone name | Heavy chain amino acid sequence |
|---|---|
| zuH3 (SEQ ID NO: | QVQLVQSGAEVKKPGASVKLSCKASGFSFTTYWITWVR |
| 2) | QAPGQGLEWIGMIHPSDSETRLSQTFKDKATLTADKST |
| | NTAYMELSSLRSEDTAVYYCARDFDVWGQGTLVTVSS |
| zuH3a (SEQ ID | QVQLVQSGAEVKKPGASVKLSCKASGFSFTTYWITWVR |
| NO: 4) | QAPGQGLEWIGMIHPSDAETRLSQTFKDKATLTADKST |
| | NTAYMELSSLRSEDTAVYYCARDFDVWGQGILVTVSS |
| zuH3b (SEQ ID | QVQLVQSGAEVKKPGASVKVSCKASGFSFTTYNITWVR |
| NO: 5) | QAPGQGLEWIGMIHPSDAETRLSQTFKDKATLTADKST |
| | NTAYMELSSLRSEDTAVYYCARDFDVWGQGTLVTVSS |
| zuH3c (SEQ ID | QVQLVQSGAEVKKPGASVKVSCKASGFSFTTYWITWVR |
| NO: 6) | QAPGQGLEWIGMIHPSDAETRLSQTFKDKATLTADKST |
| | STAYMELSSLRSEDTAVYYCARDFDVWGQGTLVTVSS |
| zuH3e (SEQ ID | QVQLVQSGAEVKKPGASVKVSCKASGFHFTTYWITWVR |
| NO: 8) | QAPGQGLEWIGMIHPSDAETRLSQTFKDKATLTADKST |
| | NTAYMELSSLRSEDTAVYYCARDFDVWGQGTLVTVSS |
| zuH3f (SEQ ID | QVQLVQSGAEVKKPGASVKVSCKASGFSFRTYWITWVR |
| NO: 9) | QAPGQGLEWIGMIHPSDAETRLSQTFKDKATLTADKST |
| | NTAYMELSSLRSEDTAVYYCARDFDVWGQGTLVTVSS |
| zuH5 (SEQ ID NO: | QVQLVQSGAEVKKPGASVKLSCKASGFSFTTYWITWVR |
| 3) | QAPGQGLEWIGMIHPSDSETRLSQTFKDKATLTADEST |
| | NTAYMELSSLRSEDTAVYYCARDFDVNGQGTLVTVSS |
| Clone name | Light chain amino acid sequence |
| zuL3 (SEQ ID NO: | DVVMTQTPLSLPVTLGQPASISCRSSQNIVHNNGNTYL |
| 12) | GWYLQKPGQSPQLLIFKVSNRFSGVPDRFSGSGSGTDF |
| | TLKISRVEAEDVGVYYCFQGSHIPRTFGQGTRLEIK |
| zuL4 (SEQ ID NO: | DVVMTQTPLSLPVTPGEPASISCRSSQNIVHNNGNTYL |
| 13) | GWYLQKPGQSPQLLIFKVSNRFSGVPDRFSGSGSGTDF |
| | TLKISRVEAEDVGVYYCFQGSHIPRTFGQGTRLEIK |
| zuL5 (SEQ ID NO: | DVVMTQSPLSLPVTLGQPASISCRSSQNIVHNNGNTYL |
| 14) | GWYLQKPGQSPQLLIFKVSNRFSGVPDRFSGSGSGTDF |
| | TIKISRVEAEDVGVYYCFQGSHIPRTFGQGTRLEIK |
| zuL5a (SEQ ID NO: | DVVMTQSPLSLPVTLGQPASISCRSSQNIVHQNGNTYL |
| 15) | GWYLQKPGQSPQLLIFKVSNRFSGVPDRFSGSGSGTDF |
| | TLKISRVEAEDVGVYYCFQGSHIPRTFGQGTRLEIK |
| zuL5c (SEQ ID NO: | DVVMTQSPLSLPVTLGQPASISCRSSQNIVHNNGQTYL |
| 17) | GWYLQKPGQSPQLLIFKVSNRFSGVPDRFSGSGSGTDF |
| | TLKISRVEAEDVGVYYCFQGSHIPRTFGQGIRLEIK |
| zuL5d (SEQ ID NO: | DVVMTQSPLSLPVTLGQPASISCRSSQNIVINNGNTYL |
| 18) | GWYLQKPGQSPQLLIFRVSNRFSGVPDRESGSGSGTDF |
| | TLKISRVEAEDVGVYYCFQGSHIPRTFGQGTRLEIK |
| zuL5e (SEQ ID NO: | DVVMTQSPLSLPVTLGQPASISCRSSQNIVHQNAQTYL |
| 19) | GWYLQKPGQSPQLLIFKVSNRFSGVPDRFSGSGSGTDF |
| | TLKISRVEAEDVGVYYCFQGSHIPRTFGQGTRLEIK |
| zuL5f (SEQ ID NO: | DVVMTQSPLSLPVTLGQPASISCRSSQNIVHQNAQTYL |
| 20) | GWYLQKPGQSPQLLIFRVSNRFSGVPDRFSGSGSGTDF |
| | TLKISRVEAEDVGVYYCFQGSHIPRTFGQGTRLEIK |
| zuL5g (SEQ ID NO: | DVVMTQSPLSLPVTLGQPASISCRSSQNIVHQNGQTYL |
| 21) | GWYLQKPGQSPQLLIFRVSNRFSGVPDRFSGSGSGTDF |
| | TLKISRVEAEDVGVYYCFQGSHIPRTFGQGTRLEIK |
| zuL5i (SEQ ID NO: | DVVMTQSPLSLPVTLGQPASISCRSSQNIVHQTGQTYL |
| 23) | GWYLQKPGQSPQLLIFRVSNRFSGVPDRFSGSGSGTDF |
| | TLKISRVEAEDVGVYYCFQGSHIPRTFGQGTRLEIK |
| zuL5j (SEQ ID NO: | DVVMTQSPLSLPVILGQPASISCRSSQNIVHQNRQTYL |
| 24) | GWYLQKPGQSPQLLIFRVSNRFSGVPDRFSGSGSGTDF |
| | TLKISRVEAEDVGVYYCFQGSHIPRTFGQGTRLEIK |
| zuL5k (SEQ ID NO: | DVVMTQSPLSLPVTLGQPASISCRSSQNIVHQNAGTYL |
| 25) | GWYLQKPGQSPQLLIFRVSNRFSGVPDREFSGSGSGTDF |
| | TLKISRVEAEDVGVYYCFQGSHIPRTFGQGERLEIK |
| zuL5o (SEQ ID NO: | DVVMTQSPLSLPVTLGQPASISCRSSQNIVHQNGQTYL |
| 29) | GWYLQKPGQSPQLLIFRVSNRFSGVPDRFSGSGSGTDF |
| | TLKISRVEAEDVGVYYCFQGSHNPRTFGQGTRLEIK |
| zuL5p (SEQ ID NO: | DVVMTQSPLSLPVTLGQPASISCRSSQNIVHQNGQTYL |
| 30) | GWYLQKPGQSPQLLIFRVSNRESGVPDRFSGSGSGTDF |
| | TLKISRVEAEDVGVYYCFQGSHHPRTFGQGTRLEIK |
| zuL5q (SEQ ID NO: | DVVMTQSPLSLPVTLGQPASISCRSSQNIVHQNGQTYL |
| 31) | GWYLQKPGQSPQLLIFRVSNRFLGVPDRFSGSGSGTDF |
| | TLKISRVEAEDVGVYYCFQGSHIPRTFGQGTRLEIK |

### Production Example 2: Production of Humanized SKM9-2 Using Different Signal Sequences

Among the antibodies obtained in Production Example 1, an antibody having high antigen binding activity and many human-derived sequences (heavy chain: zuH3c, light chain: zuL5g) was used as a representative humanized SKM9-2, and the signal sequence was examined.

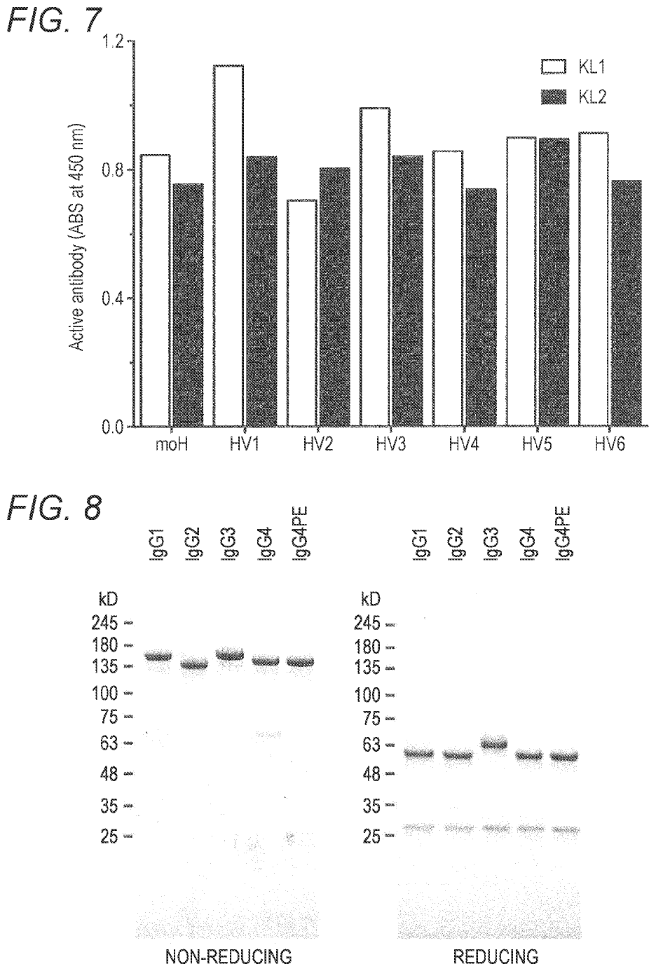

An antibody having zuH3c as the amino acid sequence of the heavy chain and zuL5g as the amino acid sequence of the light chain was produced in the same manner as in Production Example 1 except that the signal sequences shown in Table 3 were used. The results of the antibody production amount are shown in FIG. 7. No large difference was observed in the production amount of the antibody even in a case where any signal sequence was used, but when HV1 (SEQ ID NO: 221) was used as the heavy chain signal sequence and KL1 (SEQ ID NO: 227) was used as the light chain signal sequence, the production amount of the antibody tended to be slightly large.

| Sequence name | Sequence | SEQ ID NOS |
|---|---|---|
| Heavy chain signal | MDWTWRILFLVAAATGAHS | SEQ ID NO: 221 |
| sequence HV1 | | |
| Heavy chain signal | MDILCSTLLLLTVPSWVLS | SEQ ID NO: 222 |
| sequence HV2 | | |
| Heavy chain signal | MEFGLSWVFLVALLRGVQC | SEQ ID NO: 223 |
| sequence HV3 | | |
| Heavy chain signal | MKHLWFFLLLVAAPRWVLS | SEQ ID NO: 224 |
| sequence HV4 | | |
| Heavy chain signal | MGSTAILALLLAVLQGVCA | SEQ ID NO: 225 |
| sequence HV5 | | |
| Heavy chain signal | MSVSFLIFLPVLGLPWGVLS | SEQ ID NO: 226 |
| sequence HV6 | | |
| Light chain signal | MDMRVPAQLLGLLLLWVPGARC | SEQ ID NO: 227 |
| sequence KL1 | | |
| Light chain signal | MRLPAQLLGLLMLWVPGSSG | SEQ ID NO: 228 |
| sequence KL2 | | |
| Mouse heavy chain | MGWSSIILFLVATTTGVHS | SEQ ID NO: 229 |
| signal sequence | | |
| Mouse light chain | MKLPVRLLVLMFWIPASIS | SEQ ID NO: 230 |
| signal sequence | | |

### Production Example 3: Production of Humanized Antibodies of Different Subtypes

A humanized antibody was produced in the same manner as in Production Example 1 except for using zuH3c (SEQ ID NO: 6) as a heavy chain variable region, which was connected to the constant region (CH1-CH3) of human IgG, and using zuL5g as a light chain variable region, which was connected to the constant region (CL) of a human kappa chain. The IgG4PE is a variant in which two amino acid residues in the vicinity of the hinge are changed (S228P and L235E).

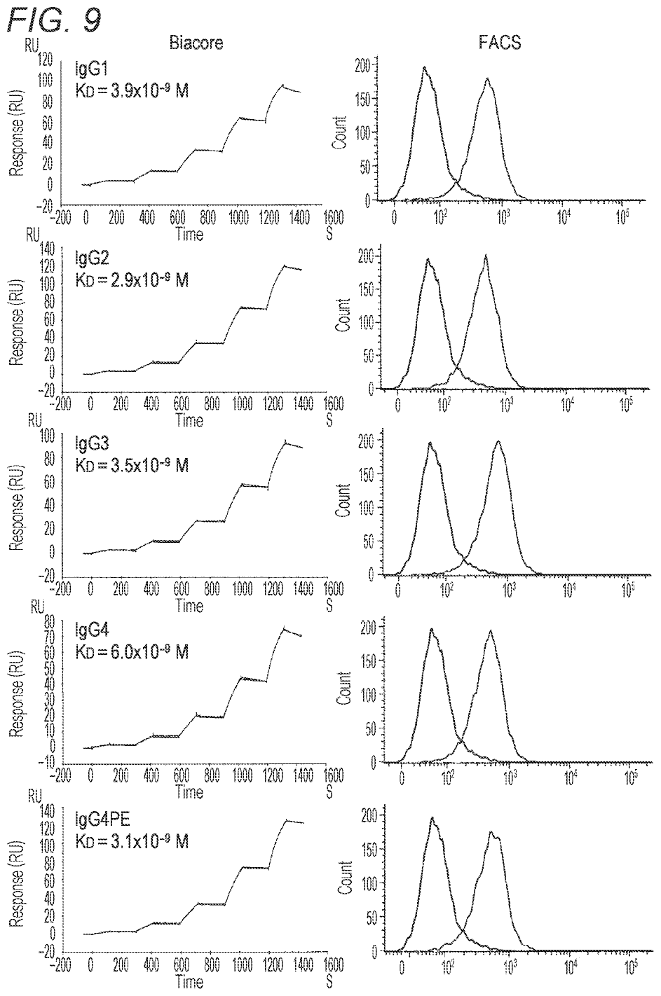

The results of SDS-PAGE of the obtained antibodies are illustrated in FIG. 8, and the results of analyzing the binding to the synthetic sugar peptide epitope represented by the following formula by Biacore X 100 and the results of analyzing the binding to mesothelioma cell line NCI-H226 by flow cytometry are illustrated in FIG. 9. Biacore used a His-tagged synthetic glycopeptide as ligand and an antibody as analyte. As the sensor chip, a sensor chip NTA bonded with Ni²⁺ was used. The affinity was calculated according to the protocol of Biacore X100. For flow cytometry, the purified antibody was used as a primary antibody at 10 μg/mL, and FITC-goat anti-human IgG F(ab)′2 was used as a secondary antibody at 25 μg/mL. The black line indicates no primary antibody (negative control), and the red line indicates addition of each primary antibody. No significant difference was observed between the antibodies for both Biacore and flow cytometry.

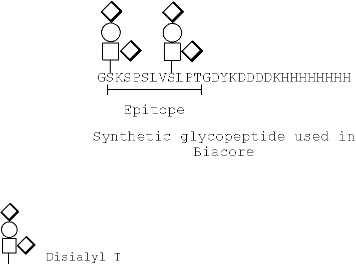

Correspondence table between sequence numbers and their contents in sequence list

| | |
|---|---|
| SEQ ID NO: 1 | xiH |
| SEQ ID NO: 2 | zuH3 |
| SEQ ID NO: 3 | zuH5 |
| SEQ ID NO: 4 | zuH3a |
| SEQ ID NO: 5 | zuH3b |
| SEQ ID NO: 6 | zuH3c |
| SEQ ID NO: 7 | zuH3d |
| SEQ ID NO: 8 | zuH3e |
| SEQ ID NO: 9 | zuH3f |
| SEQ ID NO: 10 | xiL |
| SEQ ID NO: 11 | zuL2 |
| SEQ ID NO: 12 | zuL3 |
| SEQ ID NO: 13 | zuL4 |
| SEQ ID NO: 14 | zuL5 |
| SEQ ID NO: 15 | zuL5a |
| SEQ ID NO: 16 | zuL5b |
| SEQ ID NO: 17 | zuL5c |
| SEQ ID NO: 18 | zuL5d |
| SEQ ID NO: 19 | zuL5e |
| SEQ ID NO: 20 | zuL5f |
| SEQ ID NO: 21 | zuL5g |
| SEQ ID NO: 22 | zuL5h |
| SEQ ID NO: 23 | zuL5i |
| SEQ ID NO: 24 | zuL5j |
| SEQ ID NO: 25 | zuL5k |
| SEQ ID NO: 26 | zuL5L |
| SEQ ID NO: 27 | zuL5m |
| SEQ ID NO: 28 | zuL5n |
| SEQ ID NO: 29 | zuL5o |
| SEQ ID NO: 30 | zuL5p |
| SEQ ID NO: 31 | zuL5q |
| SEQ ID NO: 32 | H-FR1 |
| SEQ ID NO: 33 | |
| SEQ ID NO: 34 | |
| SEQ ID NO: 35 | |
| SEQ ID NO: 36 | |
| SEQ ID NO: 37 | H-CDR1 |
| SEQ ID NO: 38 | |
| SEQ ID NO: 39 | |
| SEQ ID NO: 40 | H-FR2 |
| SEQ ID NO: 41 | |
| SEQ ID NO: 42 | |
| SEQ ID NO: 43 | H-CDR2 |
| SEQ ID NO: 44 | |
| SEQ ID NO: 45 | |
| SEQ ID NO: 46 | H-FR3 |
| SEQ ID NO: 47 | |
| SEQ ID NO: 48 | |
| SEQ ID NO: 49 | |
| SEQ ID NO: 50 | |
| SEQ ID NO: 51 | H-CDR3 |
| SEQ ID NO: 52 | |
| SEQ ID NO: 53 | |
| SEQ ID NO: 54 | H-FR4 |
| SEQ ID NO: 55 | |
| SEQ ID NO: 56 | |
| SEQ ID NO: 57 | L-FR1 |
| SEQ ID NO: 58 | |
| SEQ ID NO: 59 | |
| SEQ ID NO: 60 | |
| SEQ ID NO: 61 | |
| SEQ ID NO: 62 | L-CDR1 |
| SEQ ID NO: 63 | |
| SEQ ID NO: 64 | |
| SEQ ID NO: 65 | |
| SEQ ID NO: 66 | |
| SEQ ID NO: 67 | |
| SEQ ID NO: 68 | |
| SEQ ID NO: 69 | |
| SEQ ID NO: 70 | |
| SEQ ID NO: 71 | |
| SEQ ID NO: 72 | L-FR2 |
| SEQ ID NO: 73 | |
| SEQ ID NO: 74 | |
| SEQ ID NO: 75 | L-CDR2 |
| SEQ ID NO: 76 | |
| SEQ ID NO: 77 | |
| SEQ ID NO: 78 | |
| SEQ ID NO: 79 | L-FR3 |
| SEQ ID NO: 80 | |
| SEQ ID NO: 81 | |
| SEQ ID NO: 82 | L-CDR3 |
| SEQ ID NO: 83 | |
| SEQ ID NO: 84 | |
| SEQ ID NO: 85 | |
| SEQ ID NO: 86 | L-FR4 |
| SEQ ID NO: 87 | |
| SEQ ID NO: 88 | |
| SEQ ID NO: 89 | xiH-CDR1 |
| SEQ ID NO: 90 | xiH-CDR2 |
| SEQ ID NO: 91 | xiH-CDR3 |
| SEQ ID NO: 92 | zuH3-CDR1 |
| SEQ ID NO: 93 | zuH3-CDR2 |
| SEQ ID NO: 94 | zuH3-CDR3 |
| SEQ ID NO: 95 | zuH5-CDR1 |
| SEQ ID NO: 96 | zuH5-CDR2 |
| SEQ ID NO: 97 | zuH5-CDR3 |
| SEQ ID NO: 98 | zuH3a-CDR1 |
| SEQ ID NO: 99 | zuH3a-CDR2 |
| SEQ ID NO: 100 | zuH3a-CDR3 |
| SEQ ID NO: 101 | zuH3b-CDR1 |
| SEQ ID NO: 102 | zuH3b-CDR2 |
| SEQ ID NO: 103 | zuH3b-CDR3 |
| SEQ ID NO: 104 | zuH3c-CDR1 |
| SEQ ID NO: 105 | zuH3c-CDR2 |
| SEQ ID NO: 106 | zuH3c-CDR3 |
| SEQ ID NO: 107 | zuH3d-CDR1 |
| SEQ ID NO: 108 | zuH3d-CDR2 |
| SEQ ID NO: 109 | zuH3d-CDR3 |
| SEQ ID NO: 110 | zuH3e-CDR1 |
| SEQ ID NO: 111 | zuH3e-CDR2 |
| SEQ ID NO: 112 | zuH3e-CDR3 |
| SEQ ID NO: 113 | zuH3f-CDR1 |
| SEQ ID NO: 114 | zuH3f-CDR2 |
| SEQ ID NO: 115 | zuH3f-CDR3 |
| SEQ ID NO: 116 | xiL-CDR1 |
| SEQ ID NO: 117 | xiL-CDR2 |
| SEQ ID NO: 118 | xiL-CDR3 |
| SEQ ID NO: 119 | zuL2-CDR1 |
| SEQ ID NO: 120 | zuL2-CDR2 |
| SEQ ID NO: 121 | zuL2-CDR3 |
| SEQ ID NO: 122 | zuL3-CDR1 |
| SEQ ID NO: 123 | zuL3-CDR2 |
| SEQ ID NO: 124 | zuL3-CDR3 |
| SEQ ID NO: 125 | zuL4-CDR1 |
| SEQ ID NO: 126 | zuL4-CDR2 |
| SEQ ID NO: 127 | zuL4-CDR3 |
| SEQ ID NO: 128 | zuL5-CDR1 |
| SEQ ID NO: 129 | zuL5-CDR2 |
| SEQ ID NO: 130 | zuL5-CDR3 |
| SEQ ID NO: 131 | zuL5a-CDR1 |
| SEQ ID NO: 132 | zuL5a-CDR2 |
| SEQ ID NO: 133 | zuL5a-CDR3 |
| SEQ ID NO: 134 | zuL5b-CDR1 |
| SEQ ID NO: 135 | zuL5b-CDR2 |
| SEQ ID NO: 136 | zuL5b-CDR3 |
| SEQ ID NO: 137 | zuL5C-CDR1 |
| SEQ ID NO: 138 | zuL5C-CDR2 |
| SEQ ID NO: 139 | zuL5C-CDR3 |
| SEQ ID NO: 140 | zuL5d-CDR1 |
| SEQ ID NO: 141 | zuL5d-CDR2 |
| SEQ ID NO: 142 | zuL5d-CDR3 |
| SEQ ID NO: 143 | zuL5e-CDR1 |
| SEQ ID NO: 144 | zuL5e-CDR2 |
| SEQ ID NO: 145 | zuL5e-CDR3 |
| SEQ ID NO: 146 | zuL5f-CDR1 |
| SEQ ID NO: 147 | zuL5f-CDR2 |
| SEQ ID NO: 148 | zuL5f-CDR3 |
| SEQ ID NO: 149 | zuL5g-CDR1 |
| SEQ ID NO: 150 | zuL5g-CDR2 |
| SEQ ID NO: 151 | zuL5g-CDR3 |
| SEQ ID NO: 152 | zuL5h-CDR1 |
| SEQ ID NO: 153 | zuL5h-CDR2 |
| SEQ ID NO: 154 | zuL5h-CDR3 |
| SEQ ID NO: 155 | zuL5i-CDR1 |
| SEQ ID NO: 156 | zuL5i-CDR2 |
| SEQ ID NO: 157 | zuL5i-CDR3 |
| SEQ ID NO: 158 | zuL5j-CDR1 |
| SEQ ID NO: 159 | zuL5j-CDR2 |
| SEQ ID NO: 160 | zuL5j-CDR3 |
| SEQ ID NO: 161 | zuL5k-CDR1 |
| SEQ ID NO: 162 | zuL5k-CDR2 |
| SEQ ID NO: 163 | zuL5k-CDR3 |
| SEQ ID NO: 164 | zuL5L-CDR1 |
| SEQ ID NO: 165 | zuL5L-CDR2 |
| SEQ ID NO: 166 | zuL5L-CDR3 |
| SEQ ID NO: 167 | zuL5m-CDR1 |
| SEQ ID NO: 168 | zuL5m-CDR2 |
| SEQ ID NO: 169 | zuL5m-CDR3 |
| SEQ ID NO: 170 | zuL5n-CDR1 |
| SEQ ID NO: 171 | zuL5n-CDR2 |
| SEQ ID NO: 172 | zuL5n-CDR3 |
| SEQ ID NO: 173 | zuL5o-CDR1 |
| SEQ ID NO: 174 | zuL5o-CDR2 |
| SEQ ID NO: 175 | zuL5o-CDR3 |
| SEQ ID NO: 176 | zuL5p-CDR1 |
| SEQ ID NO: 177 | zuL5p-CDR2 |
| SEQ ID NO: 178 | zuL5p-CDR3 |
| SEQ ID NO: 179 | zuL5q-CDR1 |
| SEQ ID NO: 180 | zuL5q-CDR2 |
| SEQ ID NO: 181 | zuL5q-CDR3 |
| SEQ ID NO: 182 | HEGl partial |
| | peptide |
| SEQ ID NO: 183 | SLURPgpi |
| SEQ ID NO: 184 | IgG1 CH |
| SEQ ID NO: 185 | IgG1 hinge region |
| SEQ ID NO: 186 | IgG4 hinge region |
| SEQ ID NO: 187 | IgG4PE hinge |
| | region |
| SEQ ID NO: 188 | IgG1 CH2 region |
| SEQ ID NO: 189 | IgG4 or IgG4PE |
| | CH2 region |
| SEQ ID NO: 190 | Heavy chain |
| | signal sequence |
| | VH1 |
| SEQ ID NO: 191 | Heavy chain |
| | signal sequence |
| | VH2 |
| SEQ ID NO: 192 | Heavy chain |
| | signal sequence |
| | VH3 |
| SEQ ID NO: 193 | Heavy chain |
| | signal sequence |
| | VH4 |
| SEQ ID NO: 194 | Heavy chain |
| | signal sequence |
| | VH5 |
| SEQ ID NO: 195 | Heavy chain |
| | signal sequence |
| | VH6 |
| SEQ ID NO: 196 | Light chain |
| | signal sequence |
| | KL1 |
| SEQ ID NO: 197 | Light chain |
| | signal sequence |
| | KL2 |
| SEQ ID NO: 198 | Mouse heavy chain |
| | signal sequence |
| SEQ ID NO: 199 | Mouse light chain |
| | signal sequence |

1. An antibody or an antigen-binding fragment thereof, wherein
the antibody is a humanized antibody capable of binding to a human HEG1 protein expressed on mesothelioma cells, and
the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 62, a light chain CDR2 having an amino acid sequence set forth in SEQ ID NO: 75, and a light chain CDR3 having an amino acid sequence set forth in SEQ ID NO: 82.

2. The antibody or the antigen-binding fragment thereof according to claim 1, wherein the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 63, a light chain CDR2 having an amino acid sequence set forth in SEQ ID NO: 75, and a light chain CDR3 having an amino acid sequence set forth in SEQ ID NO: 82.

3. The antibody or the antigen-binding fragment thereof according to claim 1, wherein
the heavy chain variable region includes:
(1) a heavy chain CDR1 having an amino acid sequence set forth in SEQ ID NO: 89, a heavy chain CDR2 having an amino acid sequence set forth in SEQ ID NO: 90, and a heavy chain CDR3 having an amino acid sequence set forth in SEQ ID NO: 91;
(2) a heavy chain CDR1 having an amino acid sequence set forth in SEQ ID NO: 92, a heavy chain CDR2 having an amino acid sequence set forth in SEQ ID NO: 93, and a heavy chain CDR3 having an amino acid sequence set forth in SEQ ID NO: 94;
(3) a heavy chain CDR1 having an amino acid sequence set forth in SEQ ID NO: 95, a heavy chain CDR2 having an amino acid sequence set forth in SEQ ID NO: 96, and a heavy chain CDR3 having an amino acid sequence set forth in SEQ ID NO: 97;
(4) a heavy chain CDR1 having an amino acid sequence set forth in SEQ ID NO: 98, a heavy chain CDR2 having an amino acid sequence set forth in SEQ ID NO: 99, and a heavy chain CDR3 having an amino acid sequence set forth in SEQ ID NO: 100;
(5) a heavy chain CDR1 having an amino acid sequence set forth in SEQ ID NO: 101, a heavy chain CDR2 having an amino acid sequence set forth in SEQ ID NO: 102, and a heavy chain CDR3 having an amino acid sequence set forth in SEQ ID NO: 103;
(6) a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106;
(7) a heavy chain CDR1 having an amino acid sequence set forth in SEQ ID NO: 110, a heavy chain CDR2 having an amino acid sequence set forth in SEQ ID NO: 111, and a heavy chain CDR3 having an amino acid sequence set forth in SEQ ID NO: 112; or
(8) a heavy chain CDR1 having an amino acid sequence set forth in SEQ ID NO: 113, a heavy chain CDR2 having an amino acid sequence set forth in SEQ ID NO: 114, and a heavy chain CDR3 having an amino acid sequence set forth in SEQ ID NO: 115.

4. The antibody or the antigen-binding fragment thereof according to claim 1, wherein
the light chain variable region includes:
(1) a light chain CDR1 having an amino acid sequence set forth in SEQ ID NO: 116, a light chain CDR2 having an amino acid sequence set forth in SEQ ID NO: 117, and a light chain CDR3 having an amino acid sequence set forth in SEQ ID NO: 118;
(2) a light chain CDR1 having an amino acid sequence set forth in SEQ ID NO: 119, a light chain CDR2 having an amino acid sequence set forth in SEQ ID NO: 120, and a light chain CDR3 having an amino acid sequence set forth in SEQ ID NO: 121;
(3) a light chain CDR1 having an amino acid sequence set forth in SEQ ID NO: 122, a light chain CDR2 having an amino acid sequence set forth in SEQ ID NO: 123, and a light chain CDR3 having an amino acid sequence set forth in SEQ ID NO: 124;
(4) a light chain CDR1 having an amino acid sequence set forth in SEQ ID NO: 128, a light chain CDR2 having an amino acid sequence set forth in SEQ ID NO: 129, and a light chain CDR3 having an amino acid sequence set forth in SEQ ID NO: 130;
(5) a light chain CDR1 having an amino acid sequence set forth in SEQ ID NO: 131, a light chain CDR2 having an amino acid sequence set forth in SEQ ID NO: 132, and a light chain CDR3 having an amino acid sequence set forth in SEQ ID NO: 133;
(6) a light chain CDR1 having an amino acid sequence set forth in SEQ ID NO: 134, a light chain CDR2 having an amino acid sequence set forth in SEQ ID NO: 135, and a light chain CDR3 having an amino acid sequence set forth in SEQ ID NO: 136;
(7) a light chain CDR1 having an amino acid sequence set forth in SEQ ID NO: 137, a light chain CDR2 having an amino acid sequence set forth in SEQ ID NO: 138, and a light chain CDR3 having an amino acid sequence set forth in SEQ ID NO: 139;
(8) a light chain CDR1 having an amino acid sequence set forth in SEQ ID NO: 140, a light chain CDR2 having an amino acid sequence set forth in SEQ ID NO: 141, and a light chain CDR3 having an amino acid sequence set forth in SEQ ID NO: 142;
(9) a light chain CDR1 having an amino acid sequence set forth in SEQ ID NO: 143, a light chain CDR2 having an amino acid sequence set forth in SEQ ID NO: 144, and a light chain CDR3 having an amino acid sequence set forth in SEQ ID NO: 145;
(10) a light chain CDR1 having an amino acid sequence set forth in SEQ ID NO: 146, a light chain CDR2 having an amino acid sequence set forth in SEQ ID NO: 147, and a light chain CDR3 having an amino acid sequence set forth in SEQ ID NO: 148;
(11) a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151;
(12) a light chain CDR1 having an amino acid sequence set forth in SEQ ID NO: 152, a light chain CDR2 having an amino acid sequence set forth in SEQ ID NO: 153, and a light chain CDR3 having an amino acid sequence set forth in SEQ ID NO: 154;
(13) a light chain CDR1 having an amino acid sequence set forth in SEQ ID NO: 155, a light chain CDR2 having an amino acid sequence set forth in SEQ ID NO: 156, and a light chain CDR3 having an amino acid sequence set forth in SEQ ID NO: 157;
(14) a light chain CDR1 having an amino acid sequence set forth in SEQ ID NO: 158, a light chain CDR2 having an amino acid sequence set forth in SEQ ID NO: 159, and a light chain CDR3 having an amino acid sequence set forth in SEQ ID NO: 160;
(15) a light chain CDR1 having an amino acid sequence set forth in SEQ ID NO: 161, a light chain CDR2 having an amino acid sequence set forth in SEQ ID NO: 162, and a light chain CDR3 having an amino acid sequence set forth in SEQ ID NO: 163;
(16) a light chain CDR1 having an amino acid sequence set forth in SEQ ID NO: 173, a light chain CDR2 having an amino acid sequence set forth in SEQ ID NO: 174, and a light chain CDR3 having an amino acid sequence set forth in SEQ ID NO: 175;
(17) a light chain CDR1 having an amino acid sequence set forth in SEQ ID NO: 176, a light chain CDR2 having an amino acid sequence set forth in SEQ ID NO: 177, and a light chain CDR3 having an amino acid sequence set forth in SEQ ID NO: 178; or
(18) a light chain CDR1 having an amino acid sequence set forth in SEQ ID NO: 179, a light chain CDR2 having an amino acid sequence set forth in SEQ ID NO: 180, and a light chain CDR3 having an amino acid sequence set forth in SEQ ID NO: 181.

5. The antibody or the antigen-binding fragment thereof according to claim 1, further comprising:
any of the heavy chain variable regions defined in claim 3; and
any of the light chain variable regions defined in claim 4.

6. The antibody or the antigen-binding fragment thereof according to claim 1, wherein
the heavy chain variable region includes a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106, and
the light chain variable region includes a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151.

7. The antibody or the antigen-binding fragment thereof according to claim 1, wherein the heavy chain variable region includes a framework region 1 having an amino acid sequence set forth in SEQ ID NO: 32, a framework region 2 having an amino acid sequence set forth in SEQ ID NO: 40, a framework region 3 having an amino acid sequence set forth in SEQ ID NO: 46, and a framework region 4 having an amino acid sequence set forth in SEQ ID NO: 54.

8. The antibody or the antigen-binding fragment thereof according to claim 1, wherein the light chain variable region includes a framework region 1 having an amino acid sequence set forth in SEQ ID NO: 57, a framework region 2 having an amino acid sequence set forth in SEQ ID NO: 72, a framework region 3 having an amino acid sequence set forth in SEQ ID NO: 79, and a framework region 4 having an amino acid sequence set forth in SEQ ID NO: 86.

9. The antibody or the antigen-binding fragment thereof according to claim 1, wherein
the heavy chain variable region has an amino acid sequence selected from the group consisting of SEQ ID NOs: 1 to 6, 8, and 9, and
the light chain variable region has an amino acid sequence selected from the group consisting of SEQ ID NOs: 10, 12 to 15, 17 to 21, and 29 to 31.

10. The antibody or the antigen-binding fragment thereof according to claim 1, wherein
the heavy chain variable region has an amino acid sequence set forth in SEQ ID NO: 6, and
the light chain variable region has an amino acid sequence set forth in SEQ ID NO: 21.

11. A composition for use in targeting a HEG1 protein expressed in mesothelioma, the composition comprising:
the antibody or the antigen-binding fragment thereof according to claim 1.

12. (canceled)

13. A method of targeting a HEG1 protein expressed in mesothelioma in a subject, the method comprising:
injecting the subject with an effective amount of a composition including the antibody or the antigen-binding fragment thereof according to claim 1.
